(19) United States
(12) Patent Application Publication
(10) Pub. No.: US 2020/0090787 A1
(43) Pub. Date: Mar. 19, 2020

(54) SYSTEMS AND METHODS FOR SINGLE-CELL RNA-SEQ DATA ANALYSIS

(71) Applicant: AMPEL BioSolutions, LLC, Charlottesville, VA (US)

(72) Inventors: Peter E. LIPSKY, Charlottesville, VA (US); Brian KEGERREIS, Durham, NC (US); Amrie C. GRAMMER, Charlottesville, VA (US)

(21) Appl. No.: 16/557,301

(22) Filed: Aug. 30, 2019

Related U.S. Application Data
(60) Provisional application No. 62/725,753, filed on Aug. 31, 2018.

(51) Int. Cl. G16B 30/20; G16B 40/10; G16H 50/30; G16B 40/30; G16B 20/20; G16H 50/20; G16H 50/50; G06N 20/10

(52) U.S. Cl. CPC G16B 30/20; G16B 40/10; G16H 50/30; G06N 20/10; G16B 20/20; G16H 50/20; G16H 50/50; G16B 40/30

(57) ABSTRACT

Disclosed are computer-implemented methods, systems, and media for clustering cells using gene differential expression of single cells. In an aspect, a method may comprise: mapping RNA-Seq data of a plurality of cells onto a sphere (e.g., a hypersphere); calculating a plurality of distances, each of which is associated with an angle between two different cells mapped onto the sphere; clustering the plurality of cells into two clusters based on the plurality of distances; evaluating each of the two clusters using a pre-determined stopping criterion; and repeating the clustering and evaluating on each of the two clusters until the pre-determined stopping criterion or a second stopping criterion is met.

### CROSS-REFERENCE

This application claims the benefit of U.S. Provisional Patent Application No. 62/725,753, filed on Aug. 31, 2018, the contents of which are hereby incorporated in their entirety.

### BACKGROUND OF THE INVENTION

Conventional methods for quantifying molecular states of cells such as standard RNA sequencing (RNA-Seq) analysis may use an average signal of multiple cells. Single-cell RNA sequencing (scRNA-Seq) can provide the gene expression profile of individual cells.

### SUMMARY OF THE INVENTION

An average signal of a cell population obtained from conventional sequencing technologies may overlook heterogeneity within a cell population, which can be crucial for analysis of tissue functions and disease progression. Single-cell RNA sequencing (scRNA-Seq) may provide the capability to identify different cell types within a cell population, thereby allowing researchers or clinicians to characterize the subpopulation structure and function of a heterogeneous cell population. However, conventional single-cell sequencing techniques can suffer from low sequencing depth, low library size, and/or amplification bias, due to the small starting amounts of RNA in individual cells. The use of Unique Molecular Identifiers (UMI) or molecular barcodesto quantify transcripts may help alleviate some of these problems, while introducing its own technical challenges during downstream analysis. UMI-based approaches, which may comprise tagging RNA transcripts, can lessen issues related to amplification bias during library preparation. However, data sets generated using UMI-based approaches may have the vast majority (e.g., about 90-95% or more) of data entries, e.g., gene expression level, set to zero, which may confound conventional bioinformatics techniques and those designed for use with bulk RNA-Seq data. For example, the large number of zero entries can make all cells look alike in analysis techniques intended to study cellular heterogeneity.

The present disclosure provides methods, systems, and media that may advantageously allow analysis of scRNA-Seq data, e.g., UMI-based scRNA-Seq data. For example, such methods, systems, and media may advantageously improve the technical fields associated with scRNA-seq data analysis. As another example, such systems, methods, and media may advantageously improve the functionality of the computer itself by enabling automatic and unsupervised clustering of a cell population without the use of any a priori knowledge of the number of cell clusters.

The methods, systems, and media of the present disclosure may automatically cluster a cell population into cluster(s) based on expression level of a number of genes. The advantages of the methods, systems, and media disclosed herein may include, but are not limited to: eliminating the need for data normalization (e.g., scaling of one or more data entries) and/or data transformation (e.g., taking a logarithm of one or more data entries) before data can be accurately analyzed or clustered. The methods, systems, and media disclosed herein also may not require data imputation, or any other data preprocessing that may model dropout rates of genes and replace zeroes of gene expression in data entries. Conventional methods working on scRNA-Seq data may require scaling, log-transformation, and/or data imputation before they can work with the scRNA-Seq data. In some embodiments, the methods, systems, and media provided herein advantageously operate in spherical coordinates, which enables analysis of raw gene transcript counts and helps to avoid the introduction of artifacts and bias. Another advantage provided by methods, systems, and media of the present disclosure is the insensitivity to differences in library size (e.g., a total number of gene transcripts detected in one cell) among cells, as mapping onto a unit sphere (e.g., hypersphere) minimizes or eliminates differences in library size between cells. Yet another advantage of the methods, systems, and media provided herein is the elimination of the requirement for a priori knowledge of the number of clusters, while the number of clusters is determined automatically by the disclosed methods, systems, and media based on one or more criteria that stops further clustering. In some embodiments, the methods, systems, and media provided herein advantageously detect and determine whether the difference in gene expression level reflects variation within a cell cluster or heterogeneity among different cell clusters. Thus, the methods, systems, and media described herein are capable of identifying meaningful clusters of cells without over-clustering the cells. In some embodiments, the methods, systems, and media provided herein advantageously provide tools for genomic characterization of different cell populations. For example, the methods, systems, and media can be used to determine the nature of tumor-infiltrating lymphocytes, the characteristics of cells in the cerebrospinal fluid (CSF) in various diseases such as multiple sclerosis, the nature of the cells infiltrating into various organs, and/or the heterogeneity of any cells in vivo (e.g., cells in affected kidney of lupus nephritis patients) or induced in vitro.

In one aspect, disclosed herein is a computer-implemented method for clustering cells using gene differential expression of single cells, the method comprising: a) mapping RNA-Seq data of a plurality of cells onto a sphere, wherein the sphere has a dimensionality based on the RNA-Seq data of the plurality of cells; b) calculating a plurality of distances, wherein each of the plurality of distances is associated with an angle between two different cells mapped onto the sphere; c) clustering the plurality of cells into two clusters based on the plurality of distances; d) evaluating each of the two clusters using a pre-determined stopping criterion; and e) repeating c) and d) on each of the two clusters until the pre-determined stopping criterion or a second stopping criterion is met.

In some embodiments, the RNA-Seq data comprises data entries of gene expression levels. In some embodiments, the RNA-Seq data is generated using unique molecular identifiers (UMI). In some embodiments, the RNA-Seq data is not UMI-based. In some embodiments, the RNA-Seq data comprises data of each single cell of the plurality of cells. In some embodiments, the RNA-Seq data of one or more cells of the plurality of cells comprise data entries that are identical to the data entries in other cells of the plurality of cells. In some embodiments, the identical data entries are more than 50%, 60%, 70%, 80%, 90%, 95%, or more than 95%, including increments therein, of the RNA-Seq data of the one or more cells. In some embodiments, the sphere is a unit hypersphere. In some embodiments, the dimensionality of the sphere is based on a number of genes in the RNA-Seq data. In some embodiments, the dimensionality of the sphere is 3, 4, 5, 6, 7, 8, 9, 10, or greater than 10. In some embodiments, mapping the RNA-Seq data of the plurality of cells onto the sphere is based on gene expression levels. In some embodiments, mapping the RNA-Seq data of the plurality of cells onto the sphere comprises normalization of the RNA-Seq data of each of the plurality of cells by a corresponding Euclidean length thereof. In some embodiments, each of the plurality of distances is 1-cos(0), and 0 is the angle between two different cells. In some embodiments, clustering the plurality of cells into the two clusters comprises clustering the plurality of cells into only two clusters. In some embodiments, the pre-determined stopping criterion is user-defined or user-selected. In some embodiments, the pre-determined stopping criterion is automatically determined by the computer. In some embodiments, the pre-determined stopping criterion comprises a minimum cluster size, a number of genes that are differently expressed between two different clusters, a clustering silhouette, or a combination thereof. In some embodiments, the minimum cluster size is about 5, 6, 8, 10, 12, 15, 20, 50, or more than 50 cells, including increments therein. In some embodiments, the number of genes that are differently expressed between the two different clusters is about 50, 60, 70, 80, 90, 100, 120, 140, 150, 200, or more than 200, including increments therein. In some embodiments, the method further comprises receiving the RNA-Seq data of the plurality of cells, prior to a). In some embodiments, the method further comprises filtering one or more pre-determined genes from the RNA-Seq data, prior to a). In some embodiments, the method further comprises filtering one or more genes from the RNA-Seq data based on expression levels thereof, prior to a). The method of any one of the preceding claims further comprising filtering one or more genes from the RNA-Seq data based on detection rates thereof in the plurality of cells, prior to a). In some embodiments, the method further comprises filtering one or more cells from the RNA-Seq data based on a number of RNA-Seq transcripts detected, a number of genes detected, or proportion of mitochondrial transcripts detected, prior to a). In some embodiments, the method further comprises visualizing the two clusters in c), e), or both on a three-dimensional sphere. In some embodiments, the method further comprises determining one or more genes that distinguish different clusters or different groups of clusters, subsequent to e). In some embodiments, determining the one or more genes comprises using a Mann-Whitney U test. In some embodiments, the method further comprises receiving configuration data from a user for one or more parameters, prior to a). In some embodiments, the one or more parameters comprise one or more of: a minimum cluster size and a p-value for gene expression differentiation. In some embodiments, the RNA-Seq data of the plurality of cells is not normalized, prior to a). In some embodiments, the RNA-Seq data of the plurality of cells is not transformed, prior to a). In some embodiments, the RNA-Seq data of the plurality of cells is transformed by term frequency-inverse document frequency (TF-IDF), prior to a). In some embodiments, the RNA-Seq data of the plurality of cells is not subjected to imputation, prior to a). In some embodiments, the method does not use a priori knowledge of a number of clusters of the plurality of cells. In some embodiments, the RNA-Seq data of the plurality of cells comprises scRNA-Seq data. In some embodiments, the method further comprises after e), identifying a cell type from among the two clusters. In some embodiments, the cell type is classical monocytes, intermediate monocytes, non-classical monocytes, dendritic cells, B cells, T cells, plasma cells, CD4 T cells, CD8 T cells, NK cells, or NKT cells. In some embodiments, the method further comprises identifying a number of cells of the cell type. In some embodiments, the method further comprises identifying a plurality of cell types from among the two clusters. In some embodiments, the method further comprises determining a p-value for the identification of the cell type. In some embodiments, the plurality of cells is obtained from a biological sample of a subject. In some embodiments, the biological sample is obtained from an organ of the subject. In some embodiments, the organ is a kidney, pancreas, liver, lung, heart, brain, large intestine, small intestine, gallbladder, bile duct, spleen, bladder, prostate, testis, ovary, cervix, lymph node, adrenal gland, salivary gland, bone marrow, or skin. In some embodiments, the biological sample is obtained by fine needle aspiration (FNA) or biopsy of the subject. In some embodiments, the method further comprises prior to a), sequencing the plurality of cells to obtain the RNA-Seq data. In some embodiments, the method further comprises identifying a presence or absence of a disease or disorder of the subject based on the identified clusters. In some embodiments, the disease or disorder is systemic lupus erythematosus (SLE), lupus nephritis (LN), LN glomerulus, or LN tubulointerstitium. In some embodiments, the method further comprises determining a kidney disease classification of the subject based on the identified clusters. In some embodiments, the method further comprises determining a glomerular activity index of the subject based on the identified clusters. In some embodiments, the identified clusters comprise one or more of: leukocytes, T follicular helper (Tfh)-positive cells, T follicular helper (Tfh)-negative cells, regulatory T (Treg)-positive cells, regulatory T (Treg)-negative cells, T-bet-positive cells, and T-bet-negative cells. In some embodiments, the method further comprises clustering the plurality of cells into the two clusters with an Adjusted Rand Index (ARI) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In another aspect, disclosed herein is a system comprising: a digital processing device comprising: at least one processor, an operating system configured to perform executable instructions, a memory, and a computer program including instructions executable by the digital processing device to create an application for clustering cells using single-cell RNA-Seq data, comprising: a) a mapping module configured to map RNA-Seq data of a plurality of cells onto a sphere, wherein the sphere has a dimensionality based on the RNA-Seq data of the plurality of cells; b) a calculation module configured to calculate a plurality of distances, wherein each of the plurality of distances is associated with an angle between two different cells mapped on the sphere; c) a clustering module configured to cluster the plurality of cells into two clusters based on the plurality of distances; d) an evaluation module configured to evaluate each of the two clusters using a pre-determined stopping criterion; and e) a repeating module configured to repeat c) and d) on each of the two clusters until the pre-determined stopping criterion or a second stopping criterion is met.

In some embodiments, the RNA-Seq data comprises data entries of gene expression levels. In some embodiments, the RNA-Seq data is generated using unique molecular identifiers (UMI). In some embodiments, the RNA-Seq data is not UMI-based. In some embodiments, the RNA-Seq data comprises data of each single cell of the plurality of cells. In some embodiments, the RNA-Seq data of one or more cells of the plurality of cells comprise data entries that are identical to the data entries in other cells of the plurality of cells. In some embodiments, the identical data entries are more than 50%, 60%, 70%, 80%, 90%, 95%, or more than 95% (including increments therein) of the RNA-Seq data of the one or more cells. In some embodiments, the sphere is a unit hypersphere. In some embodiments, the dimensionality of the sphere is based on a number of genes in the RNA-Seq data. In some embodiments, the dimensionality of the sphere is 3, 4, 5, 6, 7, 8, 9, 10, or greater than 10. In some embodiments, mapping the RNA-Seq data of the plurality of cells onto the sphere is based on gene expression levels. In some embodiments, mapping the RNA-Seq data of the plurality of cells onto the sphere comprises normalization of the RNA-Seq data of each of the plurality of cells by a corresponding Euclidean length thereof. In some embodiments, each of the plurality of distances is 1-cos(0), and 0 is the angle between two different cells. In some embodiments, clustering the plurality of cells into the two clusters comprises clustering the plurality of cells into only two clusters. In some embodiments, the pre-determined stopping criterion is user-defined or user-selected. In some embodiments, the pre-determined stopping criterion is automatically determined by the computer. In some embodiments, the pre-determined stopping criterion comprises a minimum cluster size, a number of genes that are differently expressed between two different clusters, a clustering silhouette, or a combination thereof. In some embodiments, the minimum cluster size is about 5, 6, 8, 10, 12, 15, 20, 50, or more than 50 cells, including increments therein. In some embodiments, the number of genes that are differently expressed between two different clusters is about 50, 60, 70, 80, 90, 100, 120, 140, 150, 200, or more than 200, including increments therein. In some embodiments, the system further comprises a receiving module configured to receive the RNA-Seq data of the plurality of cells, prior to a). In some embodiments, the system further comprises a filtering module configured to filter one or more pre-determined genes from the RNA-Seq data, prior to a). In some embodiments, the system further comprises a second filtering module configured to filter one or more genes from the RNA-Seq data based on expression levels thereof, prior to a). In some embodiments, the system further comprises a third filtering module configured to filter one or more genes from the RNA-Seq data based on detection rates thereof in the plurality of cells, prior to a). In some embodiments, the system further comprises a fourth filtering module configured to filter one or more cells from the RNA-Seq data based on a number of RNA-Seq transcripts detected, a number of genes detected, or proportion of mitochondrial transcripts detected, prior to a). In some embodiments, the system further comprises a visualization module configured to visualize the two clusters in c), e), or both on a three-dimensional sphere. In some embodiments, the system further comprises a determination module configured to determine one or more genes that distinguish different clusters or different groups of clusters, subsequent to e). In some embodiments determining the one or more genes comprises using a Mann-Whitney U test. In some embodiments, the system further comprises a second receiving module configured to receive configuration data from a user for one or more parameters, prior to a). In some embodiments, the one or more parameters comprise: a minimum cluster size, or a p-value for gene expression differentiation. In some embodiments, the RNA-Seq data of the plurality of cells is not normalized, prior to a). In some embodiments, the RNA-Seq data of the plurality of cells is not transformed, prior to a). In some embodiments, the RNA-Seq data of the plurality of cells is transformed by term frequency-inverse document frequency (TF-IDF), prior to a). In some embodiments, the RNA-Seq data of the plurality of cells is not subjected to imputation, prior to a). In some embodiments, clustering the plurality of cells does not use a priori knowledge of a number of clusters of the plurality of cells. In some embodiments, the RNA-Seq data of the plurality of cells comprises scRNA-Seq data. In some embodiments, the system further comprises an identification module configured to identify a cell type from among the two clusters. In some embodiments, the cell type is classical monocytes, intermediate monocytes, non-classical monocytes, dendritic cells, B cells, T cells, plasma cells, CD4 T cells, CD8 T cells, NK cells, or NKT cells. In some embodiments, the system further comprises a quantification module configured to quantify a number of cells of the cell type. In some embodiments, the identification module identifies a plurality of cell types from among the two clusters. In some embodiments, the identification module determines a p-value for the identification of the cell type. In some embodiments, the plurality of cells is obtained from a biological sample of a subject. In some embodiments, the biological sample is obtained from an organ of the subject. In some embodiments, the organ is a kidney, pancreas, liver, lung, heart, brain, large intestine, small intestine, gallbladder, bile duct, spleen, bladder, prostate, testis, ovary, cervix, lymph node, adrenal gland, salivary gland, bone marrow, or skin. In some embodiments, the biological sample is obtained by fine needle aspiration (FNA) or biopsy of the subject. In some embodiments, the system further comprises a sequencing module configured to sequence the plurality of cells to obtain the RNA-Seq data. In some embodiments, the system further comprises a second identification module configured to identify a presence or absence of a disease or disorder of the subject based on the identified clusters. In some embodiments, the disease or disorder is systemic lupus erythematosus (SLE), lupus nephritis (LN), LN glomerulus, or LN tubulointerstitium. In some embodiments, the second identification module determines a kidney disease classification of the subject based on the identified clusters. In some embodiments, the second identification module determines a glomerular activity index of the subject based on the identified clusters. In some embodiments, the identified clusters comprise one or more of: leukocytes, T follicular helper (Tfh)-positive cells, T follicular helper (Tfh)-negative cells, regulatory T (Treg)-positive cells, regulatory T (Treg)-negative cells, T-bet-positive cells, and T-bet-negative cells. In some embodiments, the clustering module clusters the plurality of cells into the two clusters with an Adjusted Rand Index (ARI) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In yet another aspect, disclosed herein is a non-transitory computer-readable storage medium encoded with a computer program including instructions executable by a processor to create an application for clustering cells using single-cell RNA-Seq data, comprising: a) a mapping module configured to map RNA-Seq data of a plurality of cells onto a sphere, wherein the sphere has a dimensionality based on the RNA-Seq data of the plurality of cells; b) a calculation module configured to calculate a plurality of distances, wherein each of the plurality of distances is associated with an angle between two different cells mapped on the sphere; c) a clustering module configured to cluster the plurality of cells into two clusters based on the plurality of distances; d) an evaluation module configured to evaluate each of the two clusters using a pre-determined stopping criterion; and e) a repeating module configured to repeat c) and d) on each of the two clusters until the pre-determined stopping criterion or a second stopping criterion is met.

In some embodiments, the RNA-Seq data comprises data entries of gene expression levels. In some embodiments, the RNA-Seq data is generated using unique molecular identifiers (UMI). In some embodiments, the RNA-Seq data is not UMI-based. In some embodiments, the RNA-Seq data comprises data of each single cell of the plurality of cells. In some embodiments, the RNA-Seq data of one or more cells of the plurality of cells comprise data entries that are identical to the data entries in other cells of the plurality of cells. In some embodiments, the identical data entries are more than 50%, 60%, 70%, 80%, 90%, 95%, or more than 95% of the RNA-Seq data of the one or more cells, including increments therein. In some embodiments, the sphere is a unit hypersphere. In some embodiments, the dimensionality of the sphere is based on a number of genes in the RNA-Seq data. In some embodiments, the dimensionality of the sphere is 3, 4, 5, 6, 7, 8, 9, 10, or greater than 10. In some embodiments, mapping RNA-Seq data of the plurality of cells onto the sphere is based on gene expression levels. In some embodiments, mapping the RNA-Seq data of the plurality of cells onto the sphere comprises normalization of the RNA-Seq data of each of the plurality of cells by a corresponding Euclidean length thereof. In some embodiments, each of the plurality of distances is 1−cos(θ), and θ is the angle between two different cells. In some embodiments, clustering the plurality of cells into the two clusters comprises clustering the plurality of cells into only two clusters. In some embodiments, the pre-determined stopping criterion is user-defined or user-selected. In some embodiments, the pre-determined stopping criterion is automatically determined by the computer. In some embodiments, the pre-determined stopping criterion comprises a minimum cluster size, a number of genes that are differently expressed between two different clusters, a clustering silhouette, or a combination thereof. In some embodiments, the minimum cluster size is about 5, 6, 8, 10, 12, 15, 20, 50, or more than 50 cells, including increments therein. In some embodiments, the number of genes that are differently expressed between two different clusters is about 50, 60, 70, 80, 90, 100, 120, 140, 150, 200, or more than 200, including increments therein. In some embodiments, the medium further comprises a receiving module configured to receive the RNA-Seq data of the plurality of cells, prior to a). In some embodiments, the medium further comprises a filtering module configured to filter one or more pre-determined genes from the RNA-Seq data, prior to a). In some embodiments, the medium further comprises a second filtering module configured to filter one or more genes from the RNA-Seq data based on expression levels thereof, prior to a). In some embodiments, the medium further comprises a third filtering module configured to filter one or more genes from the RNA-Seq data based on detection rates thereof in the plurality of cells, prior to a). In some embodiments, the medium further comprises a fourth filtering module configured to filter one or more cells from the RNA-Seq data based on a number of RNA-Seq transcripts detected, a number of genes detected, or proportion of mitochondrial transcripts detected, prior to a). In some embodiments, the medium further comprises a visualization module configured to visualize the two clusters in c), e), or both on a three-dimensional sphere. In some embodiments, the medium further comprises a determination module configured to determine one or more genes that distinguish different clusters or different groups of clusters, subsequent to e). In some embodiments, determining the one or more genes comprises using a Mann-Whitney U test. In some embodiments, the medium further comprises a second receiving module configured to receive configuration data from a user for one or more parameters, prior to a). In some embodiments, one or more parameters comprise: a minimum cluster size, or a p-value for gene expression differentiation. In some embodiments, the RNA-Seq data of the plurality of cells is not normalized, prior to a). In some embodiments, the RNA-Seq data of the plurality of cells is not transformed, prior to a). In some embodiments, the RNA-Seq data of the plurality of cells is transformed by term frequency-inverse document frequency (TF-IDF), prior to a). In some embodiments, the RNA-Seq data of the plurality of cells is not subjected to imputation, prior to a). In some embodiments, clustering the plurality of cells does not use a priori knowledge of a number of clusters of the plurality of cells. In some embodiments, the RNA-Seq data of the plurality of cells comprises scRNA-Seq data. In some embodiments, the medium further comprises an identification module configured to identify a cell type from among the two clusters. In some embodiments, the cell type is classical monocytes, intermediate monocytes, non-classical monocytes, dendritic cells, B cells, T cells, plasma cells, CD4 T cells, CD8 T cells, NK cells, or NKT cells. In some embodiments, the medium further comprises a quantification module configured to quantify a number of cells of the cell type. In some embodiments, the identification module identifies a plurality of cell types from among the two clusters. In some embodiments, the identification module determines a p-value for the identification of the cell type. In some embodiments, the plurality of cells is obtained from a biological sample of a subject. In some embodiments, the biological sample is obtained from an organ of the subject. In some embodiments, the organ is a kidney, pancreas, liver, lung, heart, brain, large intestine, small intestine, gallbladder, bile duct, spleen, bladder, prostate, testis, ovary, cervix, lymph node, adrenal gland, salivary gland, bone marrow, or skin. In some embodiments, the biological sample is obtained by fine needle aspiration (FNA) or biopsy of the subject. In some embodiments, the medium further comprises a sequencing module configured to sequence the plurality of cells to obtain the RNA-Seq data. In some embodiments, the medium further comprises a second identification module configured to identify a presence or absence of a disease or disorder of the subject based on the identified clusters. In some embodiments, the disease or disorder is systemic lupus erythematosus (SLE), lupus nephritis (LN), LN glomerulus, or LN tubulointerstitium. In some embodiments, the second identification module determines a kidney disease classification of the subject based on the identified clusters. In some embodiments, the second identification module determines a glomerular activity index of the subject based on the identified clusters. In some embodiments, the identified clusters comprise one or more of: leukocytes, T follicular helper (Tfh)-positive cells, T follicular helper (Tfh)-negative cells, regulatory T (Treg)-positive cells, regulatory T (Treg)-negative cells, T-bet-positive cells, and T-bet-negative cells. In some embodiments, the clustering module clusters the plurality of cells into the two clusters with an Adjusted Rand Index (ARI) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a non-limiting example of pseudo-code of a clustering algorithmin accordance with disclosed embodiments.

FIG. 2 shows a non-limiting example of clusters of peripheral blood mononuclear cells (PMBCs) in a tree-branch structure, in accordance with disclosed embodiments.

FIG. 3 shows a non-limiting example of visualization of clusters of cells in FIG. 2 on a three-dimensional sphere, in accordance with disclosed embodiments.

FIG. 4 shows a non-limiting example of a work flow for analyzing kidney cells using the systems and methods herein, in accordance with disclosed embodiments.

FIG. 5 shows a non-limiting schematic diagram of a digital processing device; in this case, a device with one or more CPUs, a memory, a communication interface, and a display.

FIG. 6 shows a non-limiting schematic diagram of a web/mobile application provision system; in this case, a system providing browser-based and/or native mobile user interfaces.

FIG. 7 shows a non-limiting schematic diagram of a cloud-based web/mobile application provision system; in this case, a system comprising an elastically load balanced, auto-scaling web server, and application server resources as well as synchronously replicated databases.

FIGS. 8A-8C show a non-limiting example of a spherical visualization of cells from lupus nephritis (LN) single-cell RNA-Seq data, in accordance with disclosed embodiments, including a left 90° view (FIG. 8A), a front view (FIG. 8B), and a right 90° view (FIG. 8C). As observed in the spherical mapping, several clusters of varying densities can be clearly seen around the surface of the sphere. 20 percent of cells are shown for clarity.

FIGS. 9A-9D show a non-limiting example of a spherical visualization of centroids of clusters identified, in accordance with disclosed embodiments, including a left 90° view (FIG. 9A), a front view (FIG. 9B), and a right 90° view (FIG. 9C) of multiple cell populations, including classical monocytes, intermediate monocytes, non-classical monocytes, dendritic cells, B cells, plasma cells, CD4 T cells, CD8 T cells, and natural killer (NK) cells/NK T (NKT) cells (as indicated in the legend of FIG. 9D).

FIGS. 10A-10C show a non-limiting example of T follicular helper (Tfh) and regulatory T (Treg) signatures appearing in lupus nephritis (LN) single-cell RNA-Seq data, in accordance with disclosed embodiments, in accordance with disclosed embodiments, including a left 90° view (FIG. 10A), a front view (FIG. 10B), and a right 90° view (FIG. 10C).

FIGS. 11A-11C shows a non-limiting example of a CD11c/T-bet SLE B cell signature appearing in lupus nephritis (LN) single-cell RNA-Seq data, in accordance with disclosed embodiments including a left 90° view (FIG. 11A), a front view (FIG. 11B), and a right 90° view (FIG. 11C).

FIGS. 12A-12B show a non-limiting example of enrichment of cluster markers in lupus nephritis (LN) glomerulus (FIG. 12A) and an associated legend of enrichment score (FIG. 12B), in accordance with disclosed embodiments, including GSVA enrichment scores of cluster marker gene lists in microarray data from kidney biopsies of lupus nephritis patients (purple) and healthy controls (gold).

FIGS. 13A-13B show a non-limiting example of associations between enrichment and subject traits (FIG. 13A) and an associated legend of statistical significance as indicated by different ranges of p-values (FIG. 13B), in accordance with disclosed embodiments, including GSVA enrichment scores of cluster marker gene lists as compared to subject traits.

FIG. 14 shows a non-limiting example of a cluster dendrogram derived from cosine dissimilarities between cluster centers from lupus nephritis (LN) single-cell RNA-Seq data, in accordance with disclosed embodiments.

FIG. 15 shows a non-limiting example of a cluster dendrogram derived from cosine dissimilarities between cluster centers from pancreas single-cell RNA-Seq data, in accordance with disclosed embodiments.

### DETAILED DESCRIPTION OF THE INVENTION

An average signal of a cell population obtained from conventional sequencing technologies may overlook heterogeneity within a cell population, which can be crucial for analysis of tissue functions and disease progression. Single-cell RNA sequencing (scRNA-Seq) may provide the capability to identify different cell types within a cell population, thereby allowing researchers or clinicians to characterize the subpopulation structure and function of a heterogeneous cell population. However, conventional single-cell sequencing techniques can encounter challenges arising from low sequencing depth, low library size, and/or amplification bias, due to the small starting amounts of RNA in individual cells. The use of Unique Molecular Identifiers (UMI) or molecular barcodes to quantify transcripts may help alleviate some of these problems, while introducing its own technical challenges during downstream analysis. UMI-based approaches, which may comprise tagging RNA transcripts, can lessen issues related to amplification bias during library preparation. However, data sets generated using UMI-based approaches may have the vast majority (e.g., about 90-95% or more) of data entries, e.g., gene expression level, set to zero, which may confound existing bioinformatics techniques and those designed for use with bulk RNA-Seq data. For example, the large number of zero entries can make all cells look alike in analysis techniques intended to study cellular heterogeneity.

The present disclosure provides methods, systems, and media that may advantageously allow analysis of scRNA-Seq data, e.g., UMI-based scRNA-Seq data. For example, such methods, systems, and media may advantageously improve the technical fields associated with scRNA-seq data analysis. As another example, such systems, methods, and media may advantageously improve the functionality of the computer itself by enabling automatic and unsupervised clustering of a cell population without the use of any a priori knowledge of the number of cell clusters.

The methods, systems, and media of the present disclosure may automatically cluster a cell population into cluster(s) based on expression level of a number of genes. The advantages of the methods, systems, and media disclosed herein may include, but are not limited to: eliminating the need for data normalization (e.g., scaling of one or more data entries) and/or data transformation (e.g., taking a logarithm of one or more data entries) before data can be accurately analyzed or clustered. The methods, systems, and media disclosed herein also may not require data imputation, or any other data preprocessing that may model dropout rates of genes and replace zeroes of gene expression in data entries. Conventional methods working on scRNA-Seq data may require scaling, log-transformation, and/or data imputation before they can work with the scRNA-Seq data. In some embodiments, the methods, systems, and media provided herein advantageously operate in spherical coordinates, which enables analysis of raw gene transcript counts and helps to avoid the introduction of artifacts and bias. Another advantage provided by methods, systems, and media of the present disclosure is the insensitivity to differences in library size (e.g., a total number of gene transcripts detected in one cell) among cells, as mapping onto a unit sphere (e.g., hypersphere) minimizes or eliminates differences in library size between cells. Yet another advantage of the methods, systems, and media provided herein is the elimination of the requirement for a priori knowledge of the number of clusters, while the number of clusters is determined automatically by the disclosed methods, systems, and media based on one or more criteria that stops further clustering. In some embodiments, the methods, systems, and media provided herein advantageously detect and determine whether the difference in gene expression level reflects variation within a cell cluster or heterogeneity among different cell clusters. Thus, the methods, systems, and media described herein are capable of identifying meaningful clusters of cells without over-clustering the cells. In some embodiments, the methods, systems, and media provided herein advantageously provide tools for genomic characterization of different cell populations. For example, the methods, systems, and media can be used to determine the nature of tumor-infiltrating lymphocytes, the characteristics of cells in the cerebrospinal fluid (CSF) in various diseases such as multiple sclerosis, the nature of the cells infiltrating into various organs, and/or the heterogeneity of any cells in vivo (e.g., cells in affected kidney of lupus nephritis patients) or induced in vitro.

In one aspect, disclosed herein is a computer-implemented method for clustering cells using gene differential expression of single cells, the method comprising: a) mapping RNA-Seq data of a plurality of cells onto a sphere, wherein the sphere has a dimensionality based on the RNA-Seq data of the plurality of cells; b) calculating a plurality of distances, wherein each of the plurality of distances is associated with an angle between two different cells mapped onto the sphere; c) clustering the plurality of cells into two clusters based on the plurality of distances; d) evaluating each of the two clusters using a pre-determined stopping criterion; and e) repeating c) and d) on each of the two clusters until the pre-determined stopping criterion or a second stopping criterion is met.

In some embodiments, the RNA-Seq data comprises data entries of gene expression levels. In some embodiments, the RNA-Seq data is generated using unique molecular identifiers (UMI). In some embodiments, the RNA-Seq data is not UMI-based. In some embodiments, the RNA-Seq data comprises data of each single cell of the plurality of cells. In some embodiments, the RNA-Seq data of one or more cells of the plurality of cells comprise data entries that are identical to the data entries in other cells of the plurality of cells. In some embodiments, the identical data entries are more than 50%, 60%, 70%, 80%, 90%, 95%, or more than 95%, including increments therein, of the RNA-Seq data of the one or more cells. In some embodiments, the sphere is a unit hypersphere. In some embodiments, the dimensionality of the sphere is based on a number of genes in the RNA-Seq data. In some embodiments, the dimensionality of the sphere is 3, 4, 5, 6, 7, 8, 9, 10, or greater than 10. In some embodiments, mapping the RNA-Seq data of the plurality of cells onto the sphere is based on gene expression levels. In some embodiments, mapping the RNA-Seq data of the plurality of cells onto the sphere comprises normalization of the RNA-Seq data of each of the plurality of cells by a corresponding Euclidean length thereof. In some embodiments, each of the plurality of distances is 1-cos(θ), and θ is the angle between two different cells. In some embodiments, clustering the plurality of cells into the two clusters comprises clustering the plurality of cells into only two clusters. In some embodiments, the pre-determined stopping criterion is user-defined or user-selected. In some embodiments, the pre-determined stopping criterion is automatically determined by the computer. In some embodiments, the pre-determined stopping criterion comprises a minimum cluster size, a number of genes that are differently expressed between two different clusters, a clustering silhouette, or a combination thereof. In some embodiments, the minimum cluster size is about 5, 6, 8, 10, 12, 15, 20, 50, or more than 50 cells, including increments therein. In some embodiments, the number of genes that are differently expressed between the two different clusters is about 50, 60, 70, 80, 90, 100, 120, 140, 150, 200, or more than 200, including increments therein. In some embodiments, the method further comprises receiving the RNA-Seq data of the plurality of cells, prior to a). In some embodiments, the method further comprises filtering one or more pre-determined genes from the RNA-Seq data, prior to a). In some embodiments, the method further comprises filtering one or more genes from the RNA-Seq data based on expression levels thereof, prior to a). The method of any one of the preceding claims further comprising filtering one or more genes from the RNA-Seq data based on detection rates thereof in the plurality of cells, prior to a). In some embodiments, the method further comprises filtering one or more cells from the RNA-Seq data based on a number of RNA-Seq transcripts detected, a number of genes detected, or proportion of mitochondrial transcripts detected, prior to a). In some embodiments, the method further comprises visualizing the two clusters in c), e), or both on a three-dimensional sphere. In some embodiments, the method further comprises determining one or more genes that distinguish different clusters or different groups of clusters, subsequent to e). In some embodiments, determining the one or more genes comprises using a Mann-Whitney U test. In some embodiments, the method further comprises receiving configuration data from a user for one or more parameters, prior to a). In some embodiments, the one or more parameters comprise one or more of: a minimum cluster size and a p-value for gene expression differentiation. In some embodiments, the RNA-Seq data of the plurality of cells is not normalized, prior to a). In some embodiments, the RNA-Seq data of the plurality of cells is not transformed, prior to a). In some embodiments, the RNA-Seq data of the plurality of cells is transformed by term frequency-inverse document frequency (TF-IDF), prior to a). In some embodiments, the RNA-Seq data of the plurality of cells is not subjected to imputation, prior to a). In some embodiments, the method does not use a priori knowledge of a number of clusters of the plurality of cells. In some embodiments, the RNA-Seq data of the plurality of cells comprises scRNA-Seq data. In some embodiments, the method further comprises after e), identifying a cell type from among the two clusters. In some embodiments, the cell type is classical monocytes, intermediate monocytes, non-classical monocytes, dendritic cells, B cells, T cells, plasma cells, CD4 T cells, CD8 T cells, NK cells, or NKT cells. In some embodiments, the method further comprises identifying a number of cells of the cell type. In some embodiments, the method further comprises identifying a plurality of cell types from among the two clusters. In some embodiments, the method further comprises determining a p-value for the identification of the cell type. In some embodiments, the plurality of cells is obtained from a biological sample of a subject. In some embodiments, the biological sample is obtained from an organ of the subject. In some embodiments, the organ is a kidney, pancreas, liver, lung, heart, brain, large intestine, small intestine, gallbladder, bile duct, spleen, bladder, prostate, testis, ovary, cervix, lymph node, adrenal gland, salivary gland, bone marrow, or skin. In some embodiments, the biological sample is obtained by fine needle aspiration (FNA) or biopsy of the subject. In some embodiments, the method further comprises prior to a), sequencing the plurality of cells to obtain the RNA-Seq data. In some embodiments, the method further comprises identifying a presence or absence of a disease or disorder of the subject based on the identified clusters. In some embodiments, the disease or disorder is systemic lupus erythematosus (SLE), lupus nephritis (LN), LN glomerulus, or LN tubulointerstitium. In some embodiments, the method further comprises determining a kidney disease classification of the subject based on the identified clusters. In some embodiments, the method further comprises determining a glomerular activity index of the subject based on the identified clusters. In some embodiments, the identified clusters comprise one or more of: leukocytes, T follicular helper (Tfh)-positive cells, T follicular helper (Tfh)-negative cells, regulatory T (Treg)-positive cells, regulatory T (Treg)-negative cells, T-bet-positive cells, and T-bet-negative cells. In some embodiments, the method further comprises clustering the plurality of cells into the two clusters with an Adjusted Rand Index (ARI) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In another aspect, disclosed herein is a system comprising: a digital processing device comprising: at least one processor, an operating system configured to perform executable instructions, a memory, and a computer program including instructions executable by the digital processing device to create an application for clustering cells using single-cell RNA-Seq data, comprising: a) a mapping module configured to map RNA-Seq data of a plurality of cells onto a sphere, wherein the sphere has a dimensionality based on the RNA-Seq data of the plurality of cells; b) a calculation module configured to calculate a plurality of distances, wherein each of the plurality of distances is associated with an angle between two different cells mapped on the sphere; c) a clustering module configured to cluster the plurality of cells into two clusters based on the plurality of distances; d) an evaluation module configured to evaluate each of the two clusters using a pre-determined stopping criterion; and e) a repeating module configured to repeat c) and d) on each of the two clusters until the pre-determined stopping criterion or a second stopping criterion is met.

In some embodiments, the RNA-Seq data comprises data entries of gene expression levels. In some embodiments, the RNA-Seq data is generated using unique molecular identifiers (UMI). In some embodiments, the RNA-Seq data is not UMI-based. In some embodiments, the RNA-Seq data comprises data of each single cell of the plurality of cells. In some embodiments, the RNA-Seq data of one or more cells of the plurality of cells comprise data entries that are identical to the data entries in other cells of the plurality of cells. In some embodiments, the identical data entries are more than 50%, 60%, 70%, 80%, 90%, 95%, or more than 95% (including increments therein) of the RNA-Seq data of the one or more cells. In some embodiments, the sphere is a unit hypersphere. In some embodiments, the dimensionality of the sphere is based on a number of genes in the RNA-Seq data. In some embodiments, the dimensionality of the sphere is 3, 4, 5, 6, 7, 8, 9, 10, or greater than 10. In some embodiments, mapping the RNA-Seq data of the plurality of cells onto the sphere is based on gene expression levels. In some embodiments, mapping the RNA-Seq data of the plurality of cells onto the sphere comprises normalization of the RNA-Seq data of each of the plurality of cells by a corresponding Euclidean length thereof. In some embodiments, each of the plurality of distances is 1-cos(θ), and θ is the angle between two different cells. In some embodiments, clustering the plurality of cells into the two clusters comprises clustering the plurality of cells into only two clusters. In some embodiments, the pre-determined stopping criterion is user-defined or user-selected. In some embodiments, the pre-determined stopping criterion is automatically determined by the computer. In some embodiments, the pre-determined stopping criterion comprises a minimum cluster size, a number of genes that are differently expressed between two different clusters, a clustering silhouette, or a combination thereof. In some embodiments, the minimum cluster size is about 5, 6, 8, 10, 12, 15, 20, 50, or more than 50 cells, including increments therein. In some embodiments, the number of genes that are differently expressed between two different clusters is about 50, 60, 70, 80, 90, 100, 120, 140, 150, 200, or more than 200, including increments therein. In some embodiments, the system further comprises a receiving module configured to receive the RNA-Seq data of the plurality of cells, prior to a). In some embodiments, the system further comprises a filtering module configured to filter one or more pre-determined genes from the RNA-Seq data, prior to a). In some embodiments, the system further comprises a second filtering module configured to filter one or more genes from the RNA-Seq data based on expression levels thereof, prior to a). In some embodiments, the system further comprises a third filtering module configured to filter one or more genes from the RNA-Seq data based on detection rates thereof in the plurality of cells, prior to a). In some embodiments, the system further comprises a fourth filtering module configured to filter one or more cells from the RNA-Seq data based on a number of RNA-Seq transcripts detected, a number of genes detected, or proportion of mitochondrial transcripts detected, prior to a). In some embodiments, the system further comprises a visualization module configured to visualize the two clusters in c), e), or both on a three-dimensional sphere. In some embodiments, the system further comprises a determination module configured to determine one or more genes that distinguish different clusters or different groups of clusters, subsequent to e). In some embodiments determining the one or more genes comprises using a Mann-Whitney U test. In some embodiments, the system further comprises a second receiving module configured to receive configuration data from a user for one or more parameters, prior to a). In some embodiments, the one or more parameters comprise: a minimum cluster size, or a p-value for gene expression differentiation. In some embodiments, the RNA-Seq data of the plurality of cells is not normalized, prior to a). In some embodiments, the RNA-Seq data of the plurality of cells is not transformed, prior to a). In some embodiments, the RNA-Seq data of the plurality of cells is transformed by term frequency-inverse document frequency (TF-IDF), prior to a). In some embodiments, the RNA-Seq data of the plurality of cells is not subjected to imputation, prior to a). In some embodiments, clustering the plurality of cells does not use a priori knowledge of a number of clusters of the plurality of cells. In some embodiments, the RNA-Seq data of the plurality of cells comprises scRNA-Seq data. In some embodiments, the system further comprises an identification module configured to identify a cell type from among the two clusters. In some embodiments, the cell type is classical monocytes, intermediate monocytes, non-classical monocytes, dendritic cells, B cells, T cells, plasma cells, CD4 T cells, CD8 T cells, NK cells, or NKT cells. In some embodiments, the system further comprises a quantification module configured to quantify a number of cells of the cell type. In some embodiments, the identification module identifies a plurality of cell types from among the two clusters. In some embodiments, the identification module determines a p-value for the identification of the cell type. In some embodiments, the plurality of cells is obtained from a biological sample of a subject. In some embodiments, the biological sample is obtained from an organ of the subject. In some embodiments, the organ is a kidney, pancreas, liver, lung, heart, brain, large intestine, small intestine, gallbladder, bile duct, spleen, bladder, prostate, testis, ovary, cervix, lymph node, adrenal gland, salivary gland, bone marrow, or skin. In some embodiments, the biological sample is obtained by fine needle aspiration (FNA) or biopsy of the subject. In some embodiments, the system further comprises a sequencing module configured to sequence the plurality of cells to obtain the RNA-Seq data. In some embodiments, the system further comprises a second identification module configured to identify a presence or absence of a disease or disorder of the subject based on the identified clusters. In some embodiments, the disease or disorder is systemic lupus erythematosus (SLE), lupus nephritis (LN), LN glomerulus, or LN tubulointerstitium. In some embodiments, the second identification module determines a kidney disease classification of the subject based on the identified clusters. In some embodiments, the second identification module determines a glomerular activity index of the subject based on the identified clusters. In some embodiments, the identified clusters comprise one or more of: leukocytes, T follicular helper (Tfh)-positive cells, T follicular helper (Tfh)-negative cells, regulatory T (Treg)-positive cells, regulatory T (Treg)-negative cells, T-bet-positive cells, and T-bet-negative cells. In some embodiments, the clustering module clusters the plurality of cells into the two clusters with an Adjusted Rand Index (ARI) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In yet another aspect, disclosed herein is a non-transitory computer-readable storage medium encoded with a computer program including instructions executable by a processor to create an application for clustering cells using single-cell RNA-Seq data, comprising: a) a mapping module configured to map RNA-Seq data of a plurality of cells onto a sphere, wherein the sphere has a dimensionality based on the RNA-Seq data of the plurality of cells; b) a calculation module configured to calculate a plurality of distances, wherein each of the plurality of distances is associated with an angle between two different cells mapped on the sphere; c) a clustering module configured to cluster the plurality of cells into two clusters based on the plurality of distances; d) an evaluation module configured to evaluate each of the two clusters using a pre-determined stopping criterion; and e) a repeating module configured to repeat c) and d) on each of the two clusters until the pre-determined stopping criterion or a second stopping criterion is met.

In some embodiments, the RNA-Seq data comprises data entries of gene expression levels. In some embodiments, the RNA-Seq data is generated using unique molecular identifiers (UMI). In some embodiments, the RNA-Seq data is not UMI-based. In some embodiments, the RNA-Seq data comprises data of each single cell of the plurality of cells. In some embodiments, the RNA-Seq data of one or more cells of the plurality of cells comprise data entries that are identical to the data entries in other cells of the plurality of cells. In some embodiments, the identical data entries are more than 50%, 60%, 70%, 80%, 90%, 95%, or more than 95% of the RNA-Seq data of the one or more cells, including increments therein. In some embodiments, the sphere is a unit hypersphere. In some embodiments, the dimensionality of the sphere is based on a number of genes in the RNA-Seq data. In some embodiments, the dimensionality of the sphere is 3, 4, 5, 6, 7, 8, 9, 10, or greater than 10. In some embodiments, mapping RNA-Seq data of the plurality of cells onto the sphere is based on gene expression levels. In some embodiments, mapping the RNA-Seq data of the plurality of cells onto the sphere comprises normalization of the RNA-Seq data of each of the plurality of cells by a corresponding Euclidean length thereof. In some embodiments, each of the plurality of distances is 1−cos(θ), and θ is the angle between two different cells. In some embodiments, clustering the plurality of cells into the two clusters comprises clustering the plurality of cells into only two clusters. In some embodiments, the pre-determined stopping criterion is user-defined or user-selected. In some embodiments, the pre-determined stopping criterion is automatically determined by the computer. In some embodiments, the pre-determined stopping criterion comprises a minimum cluster size, a number of genes that are differently expressed between two different clusters, a clustering silhouette, or a combination thereof. In some embodiments, the minimum cluster size is about 5, 6, 8, 10, 12, 15, 20, 50, or more than 50 cells, including increments therein. In some embodiments, the number of genes that are differently expressed between two different clusters is about 50, 60, 70, 80, 90, 100, 120, 140, 150, 200, or more than 200, including increments therein. In some embodiments, the medium further comprises a receiving module configured to receive the RNA-Seq data of the plurality of cells, prior to a). In some embodiments, the medium further comprises a filtering module configured to filter one or more pre-determined genes from the RNA-Seq data, prior to a). In some embodiments, the medium further comprises a second filtering module configured to filter one or more genes from the RNA-Seq data based on expression levels thereof, prior to a). In some embodiments, the medium further comprises a third filtering module configured to filter one or more genes from the RNA-Seq data based on detection rates thereof in the plurality of cells, prior to a). In some embodiments, the medium further comprises a fourth filtering module configured to filter one or more cells from the RNA-Seq data based on a number of RNA-Seq transcripts detected, a number of genes detected, or proportion of mitochondrial transcripts detected, prior to a). In some embodiments, the medium further comprises a visualization module configured to visualize the two clusters in c), e), or both on a three-dimensional sphere. In some embodiments, the medium further comprises a determination module configured to determine one or more genes that distinguish different clusters or different groups of clusters, subsequent to e). In some embodiments, determining the one or more genes comprises using a Mann-Whitney U test. In some embodiments, the medium further comprises a second receiving module configured to receive configuration data from a user for one or more parameters, prior to a). In some embodiments, one or more parameters comprise: a minimum cluster size, or a p-value for gene expression differentiation. In some embodiments, the RNA-Seq data of the plurality of cells is not normalized, prior to a). In some embodiments, the RNA-Seq data of the plurality of cells is not transformed, prior to a). In some embodiments, the RNA-Seq data of the plurality of cells is transformed by term frequency-inverse document frequency (TF-IDF), prior to a). In some embodiments, the RNA-Seq data of the plurality of cells is not subjected to imputation, prior to a). In some embodiments, clustering the plurality of cells does not use a priori knowledge of a number of clusters of the plurality of cells. In some embodiments, the RNA-Seq data of the plurality of cells comprises scRNA-Seq data. In some embodiments, the medium further comprises an identification module configured to identify a cell type from among the two clusters. In some embodiments, the cell type is classical monocytes, intermediate monocytes, non-classical monocytes, dendritic cells, B cells, T cells, plasma cells, CD4 T cells, CD8 T cells, NK cells, or NKT cells. In some embodiments, the medium further comprises a quantification module configured to quantify a number of cells of the cell type. In some embodiments, the identification module identifies a plurality of cell types from among the two clusters. In some embodiments, the identification module determines a p-value for the identification of the cell type. In some embodiments, the plurality of cells is obtained from a biological sample of a subject. In some embodiments, the biological sample is obtained from an organ of the subject. In some embodiments, the organ is a kidney, pancreas, liver, lung, heart, brain, large intestine, small intestine, gallbladder, bile duct, spleen, bladder, prostate, testis, ovary, cervix, lymph node, adrenal gland, salivary gland, bone marrow, or skin. In some embodiments, the biological sample is obtained by fine needle aspiration (FNA) or biopsy of the subject. In some embodiments, the medium further comprises a sequencing module configured to sequence the plurality of cells to obtain the RNA-Seq data. In some embodiments, the medium further comprises a second identification module configured to identify a presence or absence of a disease or disorder of the subject based on the identified clusters. In some embodiments, the disease or disorder is systemic lupus erythematosus (SLE), lupus nephritis (LN), LN glomerulus, or LN tubulointerstitium. In some embodiments, the second identification module determines a kidney disease classification of the subject based on the identified clusters. In some embodiments, the second identification module determines a glomerular activity index of the subject based on the identified clusters. In some embodiments, the identified clusters comprise one or more of: leukocytes, T follicular helper (Tfh)-positive cells, T follicular helper (Tfh)-negative cells, regulatory T (Treg)-positive cells, regulatory T (Treg)-negative cells, T-bet-positive cells, and T-bet-negative cells. In some embodiments, the clustering module clusters the plurality of cells into the two clusters with an Adjusted Rand Index (ARI) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

### Certain Terms

Unless otherwise defined, all technical terms used herein have the same meaning as commonly understood by one of ordinary skill in the art to which this invention belongs.

As used herein, the singular forms “a,” “an,” and “the” include plural references unless the context clearly dictates otherwise. Any reference to “or” herein is intended to encompass “and/or” unless otherwise stated.

As used herein, the terms “about” and “substantially” refer to an amount that is near the stated amount by about ±10%, ±5%, or ±1%, including increments therein.

RNA-Seq Data

In some embodiments, the methods, systems, and media provided herein are configured for RNA sequencing (RNA-Seq) data analysis, especially single-cell RNA-Seq (scRNA-Seq) data analysis. In some embodiments, scRNA-Seq data has the potential to provide an increased understanding of cell populations in various diseases, such as lupus and cancer. However, the phenotype of individual cells may not be available or manageable when the cell population is large, e.g., about 1,000 cells, about 5,000 cells, about 10,000 cells, or more than about 10,000 cells. In some embodiments, scRNA-Seq data is analyzed to identify cell populations or clusters computationally.

In some embodiments, scRNA-Seq data may present unique technical challenges for conventional unsupervised bioinformatics techniques. For example, monocytes, B cells, and T cells can look very different from one another, but the subpopulations of each may have differing degrees of difference. Further, conventional unsupervised clustering techniques may over-cluster one or more of monocytes, B cells, or T cells into multiple clusters, given the various degree of difference in the subpopulations.

As a result, there exists an urgent need for systems and methods that are capable of automatic clustering, even when the differences between and/or within clusters vary greatly within a data set. The systems and methods herein are configured to dynamically cluster scRNA-Seq data of various cell populations by clustering to different depths, whereas conventional methods may be confounded by one or a few clusters that are highly dissimilar to the rest of the cells.

In some embodiments, the RNA-Seq data comprises data entries of gene expression levels. In some embodiments, the RNA-Seq data is generated using unique molecular identifiers (UMIs). In some embodiments, the RNA-Seq data is not generated using UMIs. In some embodiments, the RNA-Seq data comprises RNA-Seq data of each single cell of a plurality of cells, e.g., scRNA-Seq data. In some embodiments, the RNA-Seq data of one or more cells of the plurality of cells comprises data entries that are identical to the data entries in other cells of the plurality of cells. In some embodiments, the identical data entries is about 50%, 60%, 70%, 80%, 90%, 95%, or more than 95% of the RNA-Seq data of the one or more cells. As an example, data sets generated using UMI-based approaches may have the vast majority (e.g., about 90-95% or more) of data entries set to zero, which may confound conventional bioinformatics techniques and those designed for use with bulk RNA-Seq data. For example, the large number of zero entries can make all cells look alike in analysis techniques intended to study cellular heterogeneity.

In some embodiments, the RNA-Seq data comprises raw gene expression data. In some embodiments, the RNA-Seq data for each cell includes one data entry (e.g., comprising a quantitative measure of gene expression) for each gene. For example, the data entry can range from zero to an arbitrary number that is greater than zero, e.g., 10, 100, 1,000, 10,000, etc. The data entries may be normalized or un-normalized values.

In some embodiments, each cell is associated with a unique cell identification number (ID). In some embodiments, the scRNA-Seq data of a cell is associated with the unique cell ID.

Data Pre-Processing

In some embodiments, the RNA-Seq data are pre-processed before mapping onto a sphere (e.g., a hypersphere). In some embodiments, such data pre-processing can include data filtering, data normalization, data transformation, imputation, other data manipulations, or a combination thereof.

In some embodiments, the RNA-Seq data is filtered to eliminate some of the data entries based on one or more pre-determined genes from the RNA-Seq data. In some embodiments, the one or more pre-determined genes include highly variable genes. In some embodiments, the one or more pre-determined genes include rarely detected, ubiquitous, or genes that are otherwise likely to be problematic, such as mitochondrial or ribosomal transcripts. In some embodiments, at least a portion or all of the data associated with the one or more pre-determined genes are eliminated. In some embodiments, at least a portion or all of the data associated with the one or more pre-determined genes remains for clustering. In some embodiments, the one or more genes from the RNA-Seq data are filtered based on gene expression levels thereof.

In some embodiments, the RNA-Seq data is filtered based on detection rates of genes in the plurality of cells or the cell population. For example, if a gene is present in less than a pre-determined threshold (e.g., about 1% or about 0.1%) relative to the cell population being studied, then at least a portion or all of the data for that gene is removed. In some embodiments, the RNA-Seq data is filtered based on quality metrics, such as the number of total transcripts, number of unique genes, and/or proportion of transcripts that are mitochondrial in origin (e.g., a high proportion of transcripts from mitochondria can indicate cell damage, and hence such data may be filtered out or discarded). In some embodiments, at least a portion or all of the RNA-Seq data of cells that do not satisfy one or more of such quality conditions are removed from further analysis.

In some embodiments, the RNA-Seq data of the plurality of cells is transformed by term frequency-inverse document frequency (TF-IDF), prior to mapping onto the sphere (e.g., a hypersphere). In some embodiments, the TF-IDF transformation weights genes based on how common the genes are in a particular cell and how rare the genes are across all cells among a plurality of cells. In some embodiments, the RNA-Seq data of the plurality of cells is transformed using a log-transformation, either alone or in combination with other mathematical operations of the data. For example, performing a log-transformation may comprise taking a logarithm (e.g., natural logarithm, base-10 logarithm, or base-2 logarithm) of the data.

In some embodiments, the RNA-Seq data is not normalized or scaled prior to mapping onto the sphere (e.g., a hypersphere). In some embodiments, the RNA-Seq data is not transformed using mathematical operations, e.g., a log-transformation, prior to mapping onto the hypersphere. In some embodiments, the RNA-Seq data of the plurality of cells is not subjected to imputation, prior to mapping onto the sphere (e.g., a hypersphere). In some embodiments, imputation of the RNA-Seq data is configured to model dropout rates of genes and replace zeroes with estimated values. In some embodiments, imputation makes many cells look alike when they may actually be distinct populations.

Spherical Coordinates

In some embodiments, the methods, systems, and media herein use a spherical coordinate system for the RNA-Seq data. In some embodiments, the RNA-Seq data are represented using the spherical coordinate system, such that they are mapped onto a sphere (e.g., a hypersphere). The dimensionality of the sphere may be based on a number of genes. For example, after filtering the highly variable genes, if the RNA-Seq data has 101 genes, then the dimensionality of the spherical coordinate system may be 101. In some embodiments, the dimensionality of the sphere is 3, 4, 5, 6, 7, 8, 9, 10, or any integer number greater than 10. In some embodiments, the RNA-Seq data of a plurality of cells are mapped onto a high-dimension sphere, e.g., a hypersphere. In some embodiments, the dimensionality of the hypersphere is greater than, for example, 3, 4, 5, 6, 7, 8, 9, 10, 15, 20, 25, or 30, including increments therein. In some embodiments, the dimensionality herein is equivalent to “dimension.”

In some embodiments, the sphere is a unit hypersphere. In some embodiments, cells are normalized by their Euclidean length (e.g., the square root of the sum of the squares of each gene expression measurement, xᵢ: \sqrt{Σᵢ x₁²}, which has the effect of projecting all cells onto the unit hypersphere of n-dimensions, wherein n is the number of available genes, for a single cell or for the cell population. This normalization and usage of spherical coordinates may advantageously eliminate library size effects, and can be more effective than methods that use the Euclidean distance at handling data (e.g., RNA-Seq) with many zeroes.

In some embodiments, after the cells are mapped onto a unit sphere (e.g., hypersphere) using scRNA-Seq data, an angular distance between every two different cells of the cell population is calculated. In some embodiments, the distance or angular distance herein is a cosine distance metric.

In some embodiments, the angles between two different cells of the cell population (e.g., projected in a multi-dimensional space) are used to construct a cosine distance metric: 1−cos(θ), wherein 0 is the angle between two different cells mapped onto the sphere (e.g., a hypersphere). The cosine distance metric may be indicative of the cosine similarity between two vectors (e.g., representative of cell clusters), and may range from 0 (minimum similarity) to 1 (maximum similarity). Alternatively, the cosine dissimilarity, given by cos(θ), may be used as an indicator of dissimilarity between two vectors (e.g., representative of cell clusters), and may range from 0 (minimum dissimilarity and maximum similarity) to 1 (maximum dissimilarity and minimum similarity). For example, if the angle between two vectors is 0°, the cosine similarity is 1 and the cosine dissimilarity is 0. As another example, if the angle between two vectors is 90° (e.g., the vectors are orthogonal), the cosine similarity is 0 and the cosine dissimilarity is 1. The cosine similarity may be used to measure cohesion within clusters.

Using the cosine distance metric as an indicator of the cosine similarity between two vectors may be advantageous as compared to methods that use a Euclidean distance, because even if two vectors are far apart by Euclidean distance, the vectors may still have a high cosine similarity. As another example, using the cosine similarity may advantageous have low complexity, especially for sparse vectors, since only the non-zero dimensions need to be considered. In some embodiments, the cosine distance metric is calculated as:

$$1 - \frac{A \cdot B}{‖ A ‖ ‖ B ‖}$$

where A and B are vectors representing the two different cells mapped on the unit hypersphere, and “·” represents a dot product, and “∥ ∥” represents the magnitude of a vector.

Stopping Criterion

In some embodiments, the methods, systems, and media provided herein automatically stop clustering when one or more stopping criteria are met. In some embodiments, the stopping criteria are user-defined or user-selected, and can be entered before the start of cell clustering or at different time points before stopping the cell clustering process. In some embodiments, the user selects a minimum cluster size, a threshold number of genes that is differentially expressed among two clusters, a clustering silhouette, or a combination thereof. In some embodiments, the clustering silhouette is configured to assess cluster quality, for example, by measuring the distance between clusters versus the distance dispersion of clusters within clusters. In some embodiments, the user configures the specific quality threshold for clustering silhouette. For example, the user may select a difference in distance between clusters and distance within clusters as a quality threshold. In some embodiments, the differential expression is determined by non-parametric Mann-Whitney U test(s). In some embodiments, the methods, systems, and media provided herein do not cluster beyond a lower threshold. In some embodiments, the methods, systems, and media provided herein automatically test for differential expression between two child clusters. For example, if a threshold number of genes is not differentially expressed, then the split between the two child clusters is not performed. In some embodiments, the level of differential expression between two child clusters is determined by a p-value. In some embodiments, the p-value is set as a Benj amini-Hochberg corrected False Discovery Rate (FDR) of 0.0001, 0.001, 0.01, 0.02, 0.03, 0.04, 0.05, 0.06, 0.07, 0.08, 0.09, 0.10, or more than 0.10. In some embodiments, the p-value is adjusted based on the characteristics of the cell population, for example, based on a number of genes, a type of species (e.g., p-values tend to be significantly low with mice (e.g., at least 10× lower, at least 50× lower) as opposed to humans), etc.

In some embodiments, the stopping criterion is pre-determined. In some embodiments, the stopping criterion is user-defined or user-selected. In some embodiments, the pre-determined stopping criterion is automatically determined by the computer. In some embodiments, the pre-determined stopping criterion comprises a minimum cluster size, a number of genes that are differently expressed between two different clusters, or both. In some embodiments, the minimum cluster size is about 5, 6, 8, 10, 12, 15, 20, 50, or more than 50 cells. In some embodiments, the number of genes that are differently expressed between two different clusters is about 20, 30, 50, 60, 70, 80, 90, 100, 120, 140, 150, 200, or more than 200.

Clustering

In some embodiments, the methods, systems, and media herein use a clustering algorithm to automatically partition a cell population of multiple cells into clusters. In some embodiments, the clustering algorithm includes but is not limited to one or more operations, not necessarily in the exact order: a) mapping RNA-Seq data of a cell population to be analyzed onto a sphere (e.g., a hypersphere); b) calculating an angular distance between any two different cells mapped onto the sphere (e.g., hypersphere); c) clustering a parent cluster into two child clusters based on angular distances (e.g., using spherical k-means with k equals 2); d) evaluating each of the two child clusters using one or more pre-determined stopping criteria; and e) repeating c) and d) using each of the two child clusters as the parent cluster, until one or more stopping criteria are met. In some embodiments, in the very first iteration of the clustering algorithm, the parent cluster is the entirety of the cell population to be studied. In some embodiments, in subsequent iterations of the clustering algorithm, the parent cluster is one child cluster that was portioned in previous iterations. The previous iterations may or may not be immediately prior to the current iteration.

In some embodiments, the clustering algorithm described herein is a recursive tree splitting algorithm that converts a list of cell IDs into a list of lists of cell IDs, with each list representing a branch of the tree. In some embodiments, each tree branch includes one or more clusters. In some embodiments, if no further split can occur at a branch of the tree, then the branch includes only one cluster. In some embodiments, at each step of tree branch splitting, the algorithm clusters the plurality of cells by spherical k-means to form two child clusters. In some embodiments, the recursive tree splitting algorithm always divides the remaining cells needs to be further clustered into 2 clusters. In some embodiments, the algorithm tests to see if the split may have needed to be performed or if the cells of the parent cluster may need to remain as one cluster. If the cells of the parent cluster may need remain as one cluster, this tree branch terminates (e.g., algorithm stops splitting that branch). The algorithm can then move on to the highest remaining tree branch in the tree and recursively split that branch, and repeat the splitting steps for each tree branch until every tree branch terminates and no further clustering can occur based on the stopping criterion.

In some embodiments, the clustering algorithm described herein is recursive and unsupervised. In some embodiments, the clustering algorithm described herein is automatic. In some embodiments, the clustering algorithm described herein is a dynamically perform top-down, divisive clustering on scRNA-Seq data. In some embodiments, the clustering algorithm described herein clusters a plurality of cells or a cell population into two clusters based on the angular distances for every two different cells of the cell population. In some embodiments, after clustering a parent population of cells into two child clusters, the clustering algorithm evaluates each of the two child clusters using a pre-determined stopping criterion to determine whether any further clustering of the child clusters is needed. In some embodiments, after clustering a parent population into two child clusters, the clustering algorithm evaluates each of the two child clusters using a pre-determined stopping criterion to determine whether the current clustering into two child clusters is needed. If needed, the clustering algorithm can repeat the clustering and evaluating until one or more pre-determined stopping criteria have been met on every child cluster and further clustering is no longer needed.

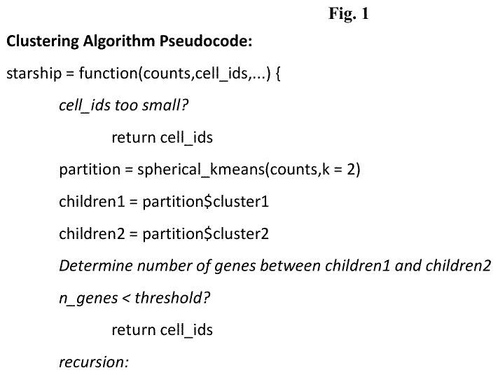

FIG. 1 shows a non-limiting example of pseudo code of a clustering algorithm, in accordance with disclosed embodiments.

In some embodiments, the clustering algorithm outputs a list of lists of cell IDs, each list of cell IDs is a tree branch representing a cell cluster, and the list of lists represent the tree with all branches. The clustering algorithm may use a spherical k means function to partition the counts into two or more clusters. Further, the clustering algorithm may determine a number of genes between a first cluster and a second cluter of the two or more clusters. The clustering algorithm may be a recursive algorithm that is performed on each of two or more children of a partitioned set of counts. The recursive algorithm may be performed until a pre-determined criterion is met. For example, the pre-determined criterion may be that the number of genes between a first cluster and a second cluter of the two or more clusters is less than a threshold.

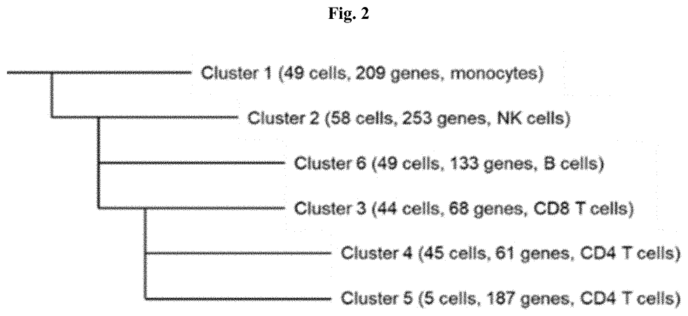

FIG. 2 shows a non-limiting example of clusters of peripheral blood mononuclear cells (PBMCs) in a tree-branch structure produced by a clustering algorithm, in accordance with disclosed embodiments. In this example, there are 6 clusters of cells produced by the clustering algorithm, and each cluster is represented by a tree branch. Two or more tree branches can be generated from a branch at a higher level. In this embodiment, cluster 1 and clusters 2-6 are partitioned apart from a same branch, and afterwards, cluster 2 and clusters 3-6 are further separated. In the next round, cluster 6 is separated from clusters 3-5. The partitioning continues until there is no further clustering that can occur at any branch or the 6 clusters.

In some embodiments, when most data entries are zero, individual cells can be represented with analogy to short documents in which many words never appear. Cells can then be clustered based on a metric, such as cosine distance, to eliminate the effect of different library sizes. The methods, systems, and media provided herein can iteratively clusters cells in a data set by spherical k-means (e.g., using k=2) to always creating, for example, two clusters at a time and testing at each step to see if partitioning along a particular branch continues. In some embodiments, the clustering algorithm provided herein does not use or require a priori knowledge about the number of clusters desired, in contrast to other conventional clustering methods. Once clustering is complete, the algorithm can test the cells in each cluster for marker genes and/or visualize the clustering to facilitate further analysis.

In some embodiments, the clustering algorithm does not apply spherical k-means to an entire cell population to be analyzed all at once. In some embodiments, the clustering algorithm herein does not use or require a priori knowledge or a well-educated estimate or guess about the number of cell types or clusters. In some embodiments, spherical k-means is applied recursively to each tree branch of data/cells as it clusters, and k is always set to 2. Applying the clustering algorithm recursively and to each tree branch of cells may advantageously allow a fine-split of cell clusters that may otherwise appear homogeneous.

In some embodiments, the clustering algorithm provided herein includes spherical multi-dimensional scaling (sMDS) to reduce the dimensionality of the sphere (e.g., hypersphere) before clustering. Instead of clustering on an in-dimensional hypersphere, where m is the number of genes in the data set, AIDS may allow clustering in n dimensions, where n can be any number (usually in the single digits, such as 2, 3, 4, 5, 6, 7, 8, or 9) that is smaller than m. In some embodiments, sMDS operates by attempting to find a low-dimensional embedding that preserves distances obtained in the high-dimensional data. Reduction of the dimension of the hypersphere may serve to reduce noise in the data set, e.g., as Principal Component Analysis (PCA).

Visualization

In some embodiments, the methods, systems, and media provided herein allows a user to visualize the clusters, e.g., the final clusters, on a three-dimensional sphere. In some embodiments, spherical multidimensional scaling (sMDS) is used to visualize samples or clusters in 3D spherical coordinates, e.g., to assess the quality of the clustering. In some embodiments, spherical multidimensional scaling (sMDS) is performed to reduce the dimensionality of the sphere (e.g., hypersphere) after clustering. Instead of an m-dimensional hypersphere, where m is the number of genes in the data set, sMDS may generate a sphere in n dimensions on which the data are mapped, where n can be any number (usually in the single digits, such as 2, 3, 4, 5, 6, 7, 8, or 9) that is smaller than m. In some embodiments, sMDS operates by attempting to find a low-dimensional embedding that preserves distances obtained in the high-dimensional data.

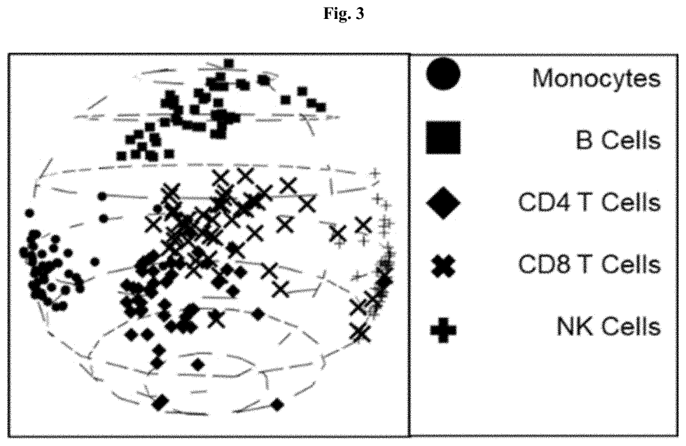

FIG. 3 shows a non-limiting example of a three-dimensional sphere mapped with scRNA-Seq data of cells for visualizing clustering of the cells, in accordance with disclosed embodiments. In this particular embodiment, 5 clusters (monocytes, B cells, CD4 T cells, CD8 T cells, and natural killer cells) are spatially separated on the three-dimensional sphere. Within each cell type/′cluster, the cells are located closely to each other than to other cluster of cells.

Cluster Analysis

In some embodiments, after the clustering has been completed for a cell population, marker genes are identified in one or more clusters, and clusters can be classified. In some embodiments, to characterize clusters, each cluster can be tested against the background of all cells to determine a list of marker genes (e.g., by Mann-Whitney U tests). In some embodiments, testing of marker genes is performed against one or more reference lists of genes to facilitate cluster identification. In some embodiments, any cluster or group of clusters can also be tested against other clusters for gene differential expression. In some embodiments, enrichment of user-defined or user-selected gene lists can be tested within the marker genes for each cluster to aid in cluster identification and/or characterization. In some embodiments, results of such tests or analysis are automatically compiled into reports to facilitate the user in quickly assessing and characterizing clusters. In some embodiments, adjusted rand index (ARI) is used to compare generated clusters to known cell types.

Digital Processing Device

In some embodiments, the methods, systems, and media described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure (e.g., a cloud-based network) that provides cloud-based computing capabilities (e.g., computational resources or storage databases). In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, handheld computers, Internet appliances, mobile smartphones, tablet computers, and personal digital assistants. In various embodiments, many smartphones are suitable for use in the system described herein. Suitable tablet computers include those with booklet, slate, and convertible configurations.

In some embodiments, the digital processing device includes an operating system configured to perform executable instructions. The operating system comprises, for example, software, including programs and data, which manages the device's hardware and provides services for execution of applications.

In some embodiments, the device includes a storage and/or memory device. The storage and/or memory device may comprise one or more physical apparatuses used to store data or programs on a temporary or permanent basis.

In some embodiments, the digital processing device includes a display configured to provide visual information to a user.

In some embodiments, the digital processing device includes an input device to receive information from a user. In some embodiments, the input device is a keyboard. In some embodiments, the input device is a pointing device including, by way of non-limiting examples, a mouse, a trackball, or a track pad. In some embodiments, the input device is a touch screen or a multi-touch screen. In other embodiments, the input device is a microphone to capture voice or other sound input.

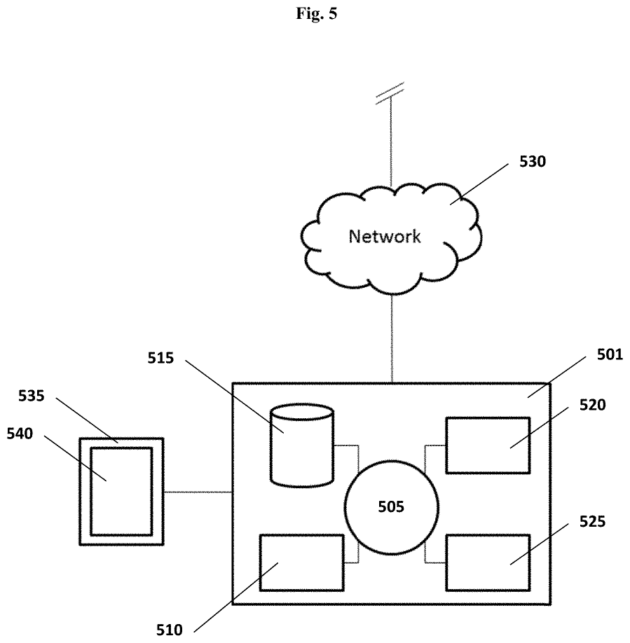

Referring to FIG. 5, in a particular embodiment, an example digital processing device 501 is programmed or otherwise configured to enable the cell clustering algorithm or application disclosed herein. The device 501 can regulate various aspects of the cell clustering application of the present disclosure, such as, for example, mapping or clustering scRNA-Seq data. In this embodiment, the digital processing device 501 includes a central processing unit (CPU, also “processor” and “computer processor” herein) 505, which can be a single-core or multi-core processor, or a plurality of processors for parallel processing. The digital processing device 501 also includes memory or memory location 110 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage, and/or electronic display adapters. The memory 510, storage unit 515, interface 520, and peripheral devices 525 are in communication with the CPU 505 through a communication bus (solid lines), such as a motherboard. The storage unit 515 can be a data storage unit (or data repository) for storing data. The digital processing device 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the device 501, can implement a peer-to-peer network, which may enable devices coupled to the device 501 to behave as a client or a server.

Continuing to refer to FIG. 5, the CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and write back. The CPU 505 can be part of a circuit, such as an integrated circuit. One or more other components of the device 501 can be included in the circuit. In some cases, the circuit is an application specific integrated circuit (ASIC) or a field programmable gate array (FPGA).

Continuing to refer to FIG. 5, the storage unit 515 can store files, such as drivers, libraries and saved programs. The storage unit 515 can store user data, e.g., user preferences and user programs. The digital processing device 501 in some cases can include one or more additional data storage units that are external, such as located on a remote server that is in communication through an intranet or the Internet.

Continuing to refer to FIG. 5, the digital processing device 501 can communicate with one or more remote computer systems through the network 530. For instance, the device 501 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®, or personal digital assistants.

Methods as described herein can be implemented by way of machine (e.g., computer processor) executable code stored on an electronic storage location of the digital processing device 101, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

Non-Transitory Computer Readable Storage Medium

In some embodiments, the methods, systems, and media disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

Computer Program

In some embodiments, the methods, systems, and media disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In various embodiments, a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

Web Application

In some embodiments, a computer program includes a web application. In various embodiments, a web application utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. In various embodiments, a web application is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

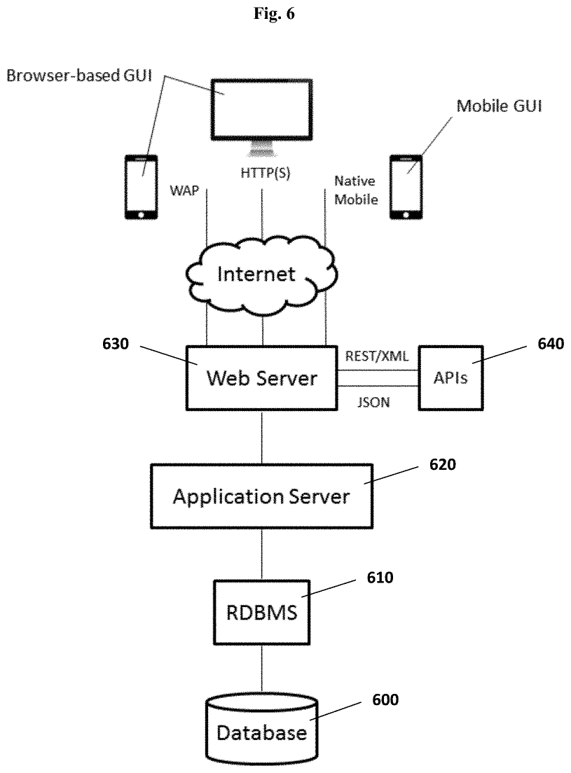

Referring to FIG. 6, in a particular embodiment, an application provision system comprises one or more databases 600 accessed by a relational database management system (RDBMS) 610. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 620 (such as Java servers, .NET servers, PHP servers, and the like) and one or more web servers 630 (such as Apache, IIS, GWS and the like). The web server(s) optionally expose one or more web services via app application programming interfaces (APIs) 640. Via a network, such as the Internet, the system provides browser-based and/or mobile native user interfaces.

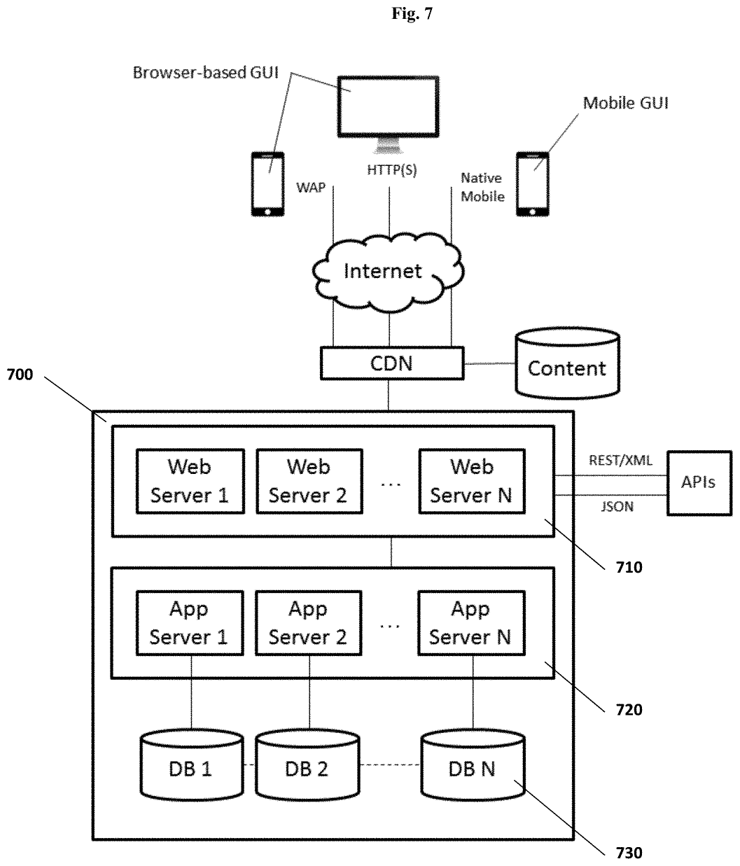

Referring to FIG. 7, in a particular embodiment, an application provision system alternatively has a distributed, cloud-based architecture 700 and comprises elastically load balanced, auto-scaling web server resources 710 and application server resources 720 as well synchronously replicated databases 730.

Software Modules

In some embodiments, the methods, systems, and media disclosed herein include software, server, and/or database modules, or use of the same. In view of the disclosure provided herein, software modules may be created by various techniques using suitable machines, software, and languages. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

Databases

In some embodiments, the methods, systems, and media disclosed herein include one or more databases, or use of the same. In various embodiments, many databases are suitable for storage and retrieval of scRNA-Seq data, cell IDs, a list of cell IDs, or a list of lists of cell IDs. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### EXAMPLES

The following illustrative examples are representative of embodiments of the software applications, systems, and methods described herein and are not meant to be limiting in any way.

### Example 1—Clustering of Peripheral Blood Mononuclear Cells (PBMCs)

According to methods and systems provided herein, 250 PBMCs (50 each of CD14 monocytes, CD19 B cells, CD4 helper T cells, CD8 T cells, and CD56 NK cells) were classified into clusters. Without using a priori knowledge of the cell types, 6 clusters were generated that closely corresponded to the known cell types, as shown in FIG. 2. A visualization of 6 clusters on a three-dimensional sphere was generated using sMDS, and shows spatial clustering of each of the cell types. For comparison, hierarchical clustering and one-off spherical k-means with k set to 5 were also performed on the same data sets, and ARI values were obtained for each approach. The hierarchical clustering produced an ARI of 0.45, the one-off spherical k-means approach produced an ARI of 0.89, and the classification according to the methods and systems provided herein produced an ARI of 0.86.

### Example 2—Comparison of Clustering of PBMCs and Mouse Embryos

The methods and systems herein were compared with four other clustering methods, including Seurat (using the Seurat R package to apply a graph-based approach for clustering), hierarchical clustering, conventional spherical k-means, and Partitioning Around Medoids (PAM) using two different data sets. One data set included 7 types of peripheral blood mononuclear cells (PBMCs) with 100 cells for each type. Another dataset included 300 mouse embryos at different development stages with 9 known types of cells. Both data sets were analyzed using all 5 clustering methods. For clustering methods other than the methods and systems disclosed herein, the data was normalized, and highly variable genes were filtered. The number of cell types was set as the known number for hierarchical clustering, conventional spherical k-means, and Partitioning Around Medoids (PAM). The Adjusted Rand Index (ARI), which measures the agreement between clusters generated using the methods and the known cell types, was calculated as a metric for comparison of the clustering methods. The systems and methods disclosed herein generated the highest ARI for both data sets among the five clustering methods tested (ARI=0.66 for PMBCs and ARI=0.52 for embryo data set). For the mouse embryo data set, 9 cell types were automatically identified, and 6 out of the 7 cell types of the PMBC were identified using the methods and systems disclosed herein. Therefore, the methods and systems disclosed herein outperformed the Seurat, hierarchical clustering, traditional spherical k-means, and Partitioning Around Medoids (PAM) approaches in performing clustering of two example data sets from PBMCs and mouse embryos.

### Example 3—Clustering of Kidney Cells in Lupus Patients

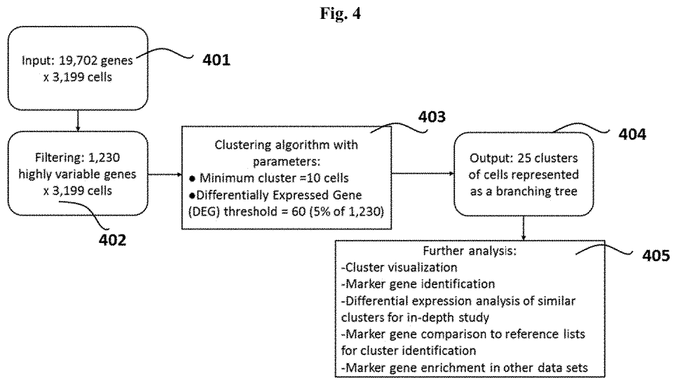

As shown in FIG. 4, in a particular embodiment, 3,199 cells were obtained from 30 patients and 5 controls 401. The cells were analyzed by scRNA-Seq. There were 19,702 genes in the cells, and all the genes were processed by gene filtering (as in operation 402). After the filtering, data associated with 1,230 variable genes remained for clustering. Genes with low detection rate or associated with mitochondrial transcripts were also filtered out. A user can configure one or more parameters for the clustering algorithm 403. In this embodiment, a user pre-set differentially expressed gene threshold to 60 genes (5% of available genes). The minimum cluster size was set by the user to be 10 cells. Using the systems and methods provided herein, 3,199 cells were automatically clustered with 1,230 genes. As a result, 25 clusters were generated as the output 404. The marker genes of each cluster were determined by Mann-Whitney U tests. These sets of marker genes were used as input to Gene Set Variation Analysis (GSVA), a separate R package that calculates enrichment scores for lists of genes. GSVA was applied to microarray data from lupus nephritis (LN) glomerulus and lupus nephritis (LN) tubulointerstitium to generate enrichment scores, which can be tested for associations with disease severity.

In this particular embodiment, analysis of microarray data from lupus nephritis (LN) glomerulus showed that nearly all leukocyte clusters identified using the systems and methods provided herein were positively associated with disease, and all non-leukocyte clusters were negatively associated with disease. After clustering was completed, the systems and methods provided herein allow one or more analysis operations (as in 405) for further analysis of the cells and genes based on the user's selection: such as cluster visualization, marker gene identification, differential expression analysis of similar clusters for in-depth study, marker gene comparison to reference lists for cluster identification, and marker gene enrichment in other data sets.

### Example 4—Analyzing Single-Cell RNA-Seq Data from Lupus Nephritis Samples Using a Spherical Transformation and Recursive Splitting for Heuristic Identification of Partitions (Starship) Approach

Using methods and systems of the present disclosure, single-cell RNA-Seq data was obtained from lupus nephritis (LN) samples and then analyzed by a clustering approach called spherical transformation and recursive splitting for heuristic identification of partitions (Starship), which is adapted for single-cell RNA-Seq data. Generally, bulk cell analysis methods may fail to account for the zero-inflated nature of single-cell RNA-Seq data. For example, Euclidean-based methods may be confounded by the vast number of zeros, which tends to make all cells look similar. In addition, density-based methods may fail to adapt to different levels of heterogeneity among leukocytes (e.g., the differences between myeloid populations may be more prominent than those between B cells and T cells). For example, conventional methods may be unable to cluster all of the cells in one pass, and may need to be re-run manually on sub-clusters to fully partition the cells.

Single-cell RNA-Seq data, particularly those gathered with Unique Molecular Identifier (UMI) barcodes, may tend to resemble bag-of-words text data in several ways, such as: 1) each observation takes an integer value, and 2) most genes may not appear in a given cell, much like most words may not appear in a given document. Clustering of this sparse data may be performed by mapping samples onto the surface of a unit n-dimensional sphere, where n is the number of genes. Rather than clustering with a set number of clusters (k), Starship recursively clusters data with k=2 until pre-defined stop criteria are met. Once the clustering is complete, several functions can be run to further analyze and/or visualize the resulting clusters of cells.

Starship was validated in two data sets with known biological information. The first data set is sorted PBMCs (as described by, for example, Zheng et al., “Massively parallel digital transcriptional profiling of single cells,” Nature Communications, Jan. 16, 2017, which is incorporated herein by reference in its entirety), which is available at support.10×genomics.com/single-cell-gene-expression/datasets. The second data set is mouse embryos (single-embryo, rather than single-cell, RNA-Seq) (as described by, for example, Deng et al., “Single-cell RNA-seq reveals dynamic, random monoallelic gene expression in mammalian cells,” Science, Jan. 10, 2014, which is incorporated herein by reference in its entirety), which is available at hemberg-lab.github.io/scRNA.seq.datasets/mouse/edev and from GEO as GSE45719. In these instances, Starship autonomously identified a reasonable number of clusters and achieved excellent agreement with known cell identities, as indicated by the Adjusted Rand Index (ARI) metric of clustering algorithms.

The StarShip clustering algorithm comprises one or more operations, including filtering cells from the data, filtering genes from the data, clustering cells from the data, and characterizing the clusters.

Cells are filtered from the data as follows. Low-quality cell data are filtered based on metrics, such as the number of UMIs per cell, the number of unique genes, the percentage of mitochondrial genes, or a combination thereof. For each of these metrics, outlier cells that fall at least 3 median absolute deviations outside the median are filtered out (e.g., removed and discarded) from the data set.

Next, genes are filtered from the data as follows. Rare genes are filtered out from the data based on a pre-determined criterion or threshold (e.g., genes appearing in less than 1%, 0.1%, or 0.01% of cells). This filtering may remove a significant portion of the genes (e.g., about 30%, about 40%, about 50%, about 60%, about 70%, about 80%, or about 90%). Next, “differentially expressed” genes are filtered out from the data. For example, this filtering may be performed using the M3Drop R package, which models RNA amplification according to Michaelis-Menten kinetics and provides a set of genes of interest (e.g., genes that are expressed differently than what the model predicts for the vast majority of genes). This filtering may result in a manageable set of genes (e.g., at most about 500, at most about 1,000, at most about 1,500, at most about 2,000, at most about 2,500, at most about 3,000, at most about 3,500, at most about 4,000, at most about 4,500, at most about 5,000, at most about 6,000, at most about 7,000, at most about 8,000, at most about 9,000, or at most about 10,000 genes).

Next, cell clustering is performed as follows. A starship( ) function is used to cluster cells. This function allows the user to specify several options, such as whether to pre-process cells using multidimensional scaling (MDS, default is FALSE) and whether MDS is based on cosine distance or geometric (e.g., Euclidean) distance. The function also allows the user to specify the clustering method (e.g., spherical k-means by default, or k-medoids), the minimum cluster size (default is 10), a heuristic to stop clustering (number of differentially expressed genes between clusters by default, or cluster silhouette), a p-value threshold for differential expression, how expression values are scaled prior to differential expression analysis (L₂ norm by default, L₁ norm, or none), the number of differentially expressed genes required between clusters (default value of 1, but can be set to another number, such as about 10, about 20, about 30, about 40, about 50, about 60, about 70, about 80, about 90, or about 100), a cluster silhouette threshold (default 0.1, but can be set to another number), and a random seed to ensure reproducible results. The function returns the tree created by the clustering process, along with the expression data used in clustering and the call to starship( ) itself to track the options that were chosen.

Next, the clusters are characterized as follows. First, a tree2list( ) function is used to collapse the tree of clustered samples down to a list with one element for each cluster. Next, a cluster assignments( ) function is used to extract the cluster assignment for each cell. Next, a test tree splits( ) function performs differential expression analysis between an experimental group comprising one or more clusters and a control group comprising one or more clusters. The user can specify how to scale expression values (L₂ norm by default, L₁ norm, or no scaling) and a p-value threshold for differential expression. If no control clusters are specified, the control group is assumed to be all cells not in the experimental group. This function returns a list of upregulated genes in the experimental group if no control group is specified, or a list of upregulated genes and downregulated genes otherwise. Next, a test all clusters( ) function applies the test tree splits( ) function to each cluster against all other clusters to acquire a list of marker genes for each cluster.

Next, to generate a visualization of the clusters, a tree( ) function plots a histogram based on the distances between cluster centers. Then, visualize clusters( ) and visualize cells by cluster( ) functions perform multidimensional scaling to map clusters or cells onto a 3D sphere, which is plotted using the plotly R package.

Next, an iscope_report( ) function performs functional enrichment based on the result of the test_all_clusters( ) function. Given a list of functional categories of genes and a test to perform (e.g., Fisher's exact test or chi-squared test), this function tests each cluster's marker genes for enrichment of functional categories, and provides test statistics and p values. It currently supports ISCOPE annotations but can work with essentially any functional annotation schema. Next, a write_reports( ) function compiles the results of the test_tree_splits( ) and iscope_report( ) functions, and creates spreadsheet files for data storage and further analysis.

### Example 5—Validating the StarShip Clustering Algorithm

The Starship clustering algorithm in validated using more varied data sets. This addresses a limitation that labeled UMI data sets are rare, which results in a lack of ground-truth data sets for use in training and/or validating clustering algorithms. For some data sets (e.g., hematopoietic stem cells or a well-characterized tissue such as pancreas), validation may simply comprise finding a reasonable number of clusters with appropriate marker genes if the cells are not labeled with known cell types.

As scRNA-Seq data is updated with more cells, more genes, and more counts per gene, this may produce challenges in parsing out unique sub-populations, such as lymphoid cell populations.

A parallel advancement in the bag-of-words-inspired space is achieved by using Latent Dirichlet Allocation to cluster cells and get marker genes in one step. Using the text2vec R package (text2vec.org) in conjunction with the LDAvis R package (cran.r-project.org/web/packages/LDAvis/index.html) allows for rapid exploration of LDA models. The LDA may be performed using various other R packages (e.g., topicmodels and lda), but the text2vec package may be faster and simpler.

### Example 6—Analyzing Single-Cell RNA-Seq Data from Lupus Nephritis Samples

Single-cell RNA-Seq (scRNA-seq) has the potential to provide a deeper understanding of cell populations in diseases and disorders, such as lupus. Diseases and disorders such as lupus nephritis (LN) may have heterogeneous gene expression across individual cells of affected subjects. Therefore, additional information about the mechanisms involved in lupus nephritis can be determined from analyzing the individual cells within the affected kidney. For example, kidney scRNA-Seq data from lupus nephritis (LN) patients provides an opportunity to determine the heterogeneity of cells within the affected kidney. However, since individual cells are not identified phenotypically, it may be necessary to identify cell populations computationally. One approach to assess the cells within an organ is to analyze the messenger RNA (mRNA) expressed by each individual cell. However, analysis of this data is challenging. Analysis of scRNA-Seq data present technical challenges that make it difficult to approach this analysis with conventional unsupervised bioinformatics techniques. Natural language processing (NLP)-inspired techniques, however, can be leveraged to identify meaningful clusters of cells without prior knowledge of the cell types present in the sample.

A clustering algorithm was developed to assess the messenger RNA expressed by each cell and, thereby, the nature of the cells in organs (e.g., the kidney) without bias. The method is a recursive, unsupervised, heuristic technique (StarShip) to dynamically perform top-down, divisive clustering on scRNA-Seq data. The method begins by mapping a plurality of cells onto an n-dimensional unit sphere, where n is the number of available genes. Next, the angles between all cells are used to construct a cosine distance metric: 1−cos(θ). The cosine distance is used to carry out k-means or k-medoids clustering, with k set to 2 for each iteration. At each split of the data, the clustering algorithm evaluates whether it has sorted the remaining cells into meaningful populations and stops making splits when a user-defined or user-selected criterion is met. Once all clusters are finalized, a Mann-Whitney U test determines genes that distinguish clusters or groups of clusters from other cells.

The clustering algorithm was validated using publicly available peripheral blood mononuclear cell (PBMC) scRNA-Seq data from 10× Genomics and tested in scRNA-Seq data from LN patients. The Adjusted Rand Index (ARI) was used to compare generated cluster partitions to known cell types in the PBMC data.

Data sources were obtained for analysis using the cluster algorithm as follows. Single cell RNA-Seq data from lupus nephritis patients was acquired from Immport: immport.org/immport-open/public/study/study/displayStudyDetail/SDY997. Single cell RNA-Seq data from sorted human PBMCs was acquired from 10× Genomics: support.10×genomics.com/single-cell-gene-expression/datasets.

Single embryo RNA-Seq data from mice was acquired from hemberg-lab.github.io/scRNA.seq.datasets (as described by Deng et al., 2014, GSE45719). Lupus nephritis microarray data (GSE32591) was acquired from GEO (as described by, for example, Berthier et al., “Cross-species transcriptional network analysis defines shared inflammatory responses in murine and human lupus nephritis,” Journal of Immunology, Jun. 20, 2012, which is incorporated herein by reference in its entirety).

Filtering of cells and genes was performed as follows. Cells were filtered based on three criteria: the number of reads, the number of unique genes, and the proportion of mitochondrial transcripts. A log-transformation was performed on these data, and outlier cells having values greater than 3 median absolute deviations from the median were removed. Genes were filtered out if they were detected in only a small minority of cells (e.g., 0.1% or 1%, depending on the number of cells). Further selection of genes for analysis was performed using the M3Drop R package, which fits mean expression levels and detection rates to a Michaelis-Menten curve in order to identify informative genes.

The StarShip clustering algorithm was performed as follows. First, the Starship algorithm maps gene expression data from all cells onto an n-dimensional unit sphere, where n is the number of genes in the data set. The angles between all cells on this sphere are used to construct a cosine distance metric, which is used to carry out spherical k-means clustering on the cells. StarShip works from the top-down, first partitioning all cells into 2 branches and then recursively splitting each branch of the tree until stopping criteria are met. At each partitioning operation, StarShip tests the resulting clusters for differentially expressed genes and stops processing the branch if there are too few genes that distinguish the clusters.

Validation metrics were determined as follows. The Adjusted Rand Index (ARI) was used to assess the agreement between cluster assignments and known cell types. The ARI has a value of zero for no agreement and a value of 1 for perfect agreement.

Markers were identified as follows. Marker genes for clusters were identified by comparing expression levels for a given cluster to those of all other cells by the Mann-Whitney U test with Benjamini-Hochberg FDR correction. Genes with FDR <0.01 were maintained as markers.

Single-cell gene signature enrichment was performed as follows. Gene signatures were tested for significant enrichment within single cells by the one-sided Kolmogorov-Smirnov test. The distribution of genes within a signature was compared to that of all other genes in the cell to determine whether the signature was enriched.

Gene set variation analysis (GSVA): The GSVA R package was used as a non-parametric, unsupervised method for estimating the variation of cluster marker gene sets in microarray gene expression data from kidney biopsies of lupus nephritis and healthy controls. Enrichment scores were calculated using a Kolmogorov-Smirnov-like random walk statistic. Enrichment scores were compared to patient traits by t-test or Spearman rank correlation, as appropriate.

The StarShip algorithm was used to classify 250 PBMCs (50 each of CD14 monocytes, CD19 B cells, CD4 helper T cells, CD8 T cells, and CD56 NK cells) into clusters. Using dynamic spherical k-means, 6 clusters were generated that closely corresponded to the known cell types (as shown in FIG. 2). For comparison, hierarchical clustering and one-off spherical k-means with k set to 5 were also performed on the same data sets, and ARI values were obtained for each approach. The hierarchical clustering produced an ARI of 0.45, the one-off spherical k-means approach produced an ARI of 0.89, and the classification according to the methods and systems provided herein produced an ARI of 0.86.

Further, data sets from labeled human PBMCs and mouse embryos were used to validate the StarShip algorithm's performance. The dynamic spherical k-means algorithm was compared to static spherical k-means, the Seurat R package, hierarchical clustering, and partitioning around medoids (PAM). The Adjusted Rand Index (ARI) was calculated as a metric for comparison of the clustering methods' results to known labels. An ARI value of 1 denotes perfect agreement, while 0 denotes no agreement. As shown in Table 1, the StarShip clustering algorithm outperforms other clustering methods.

TABLE 1
Performance comparison of the StarShip clustering
algorithm vs. four other clustering algorithms

| | Zheng Human PBMC | Deng Mouse Embryos |
|---|---|---|
| | (5 cell types) | (9 stages) |
| StarShip | 6 clusters | 9 clusters |
| | ARI: 0.86 | ARI: 0.52 |
| Static spherical | Set to k = 5 | Set to k = 9 |
| k-means | ARI: 0.89 | ARI: 0.46 |
| Seurat | 4 clusters | 5 clusters |
| | ARI: 0.69 | ARI: 0.48 |
| Hierarchical | Set to k = 5 | Set to k = 9 |
| clustering | ARI: 0.19 | ARI: 0.44 |
| Partitioning | Set to k = 5 | Set to k = 9 |
| Around Medoids | ARI: 0.46 | ARI: 0.51 |

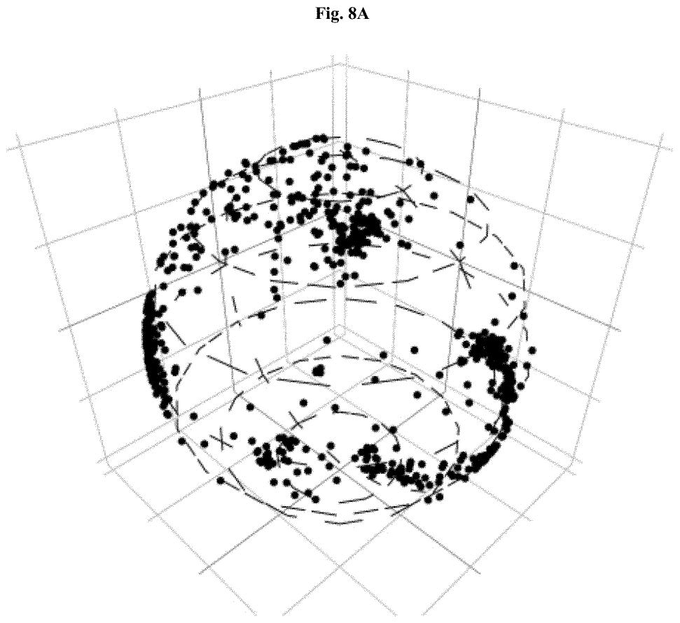

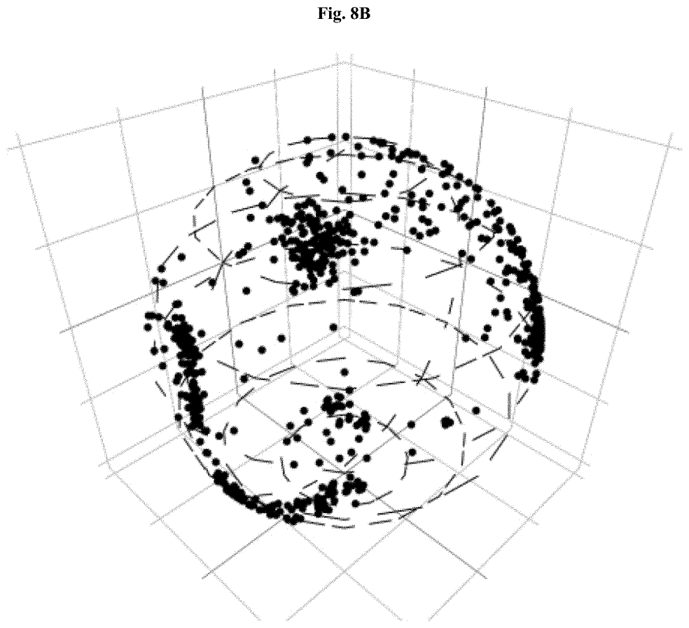

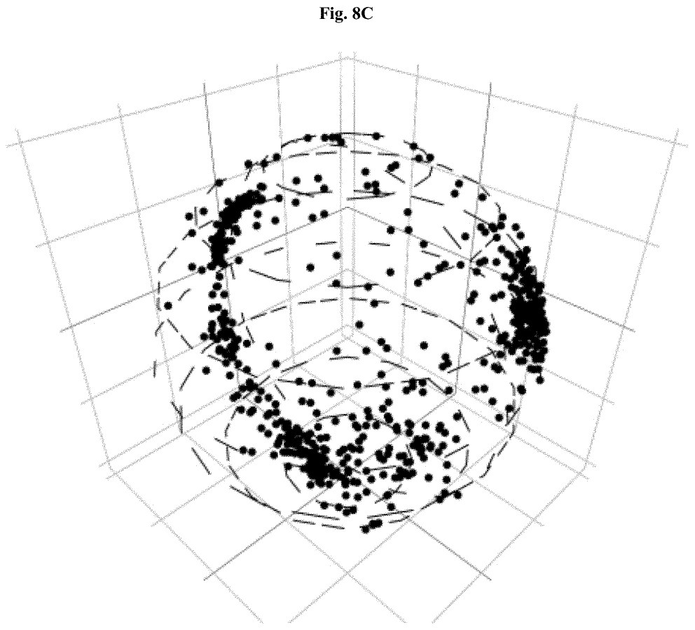

FIGS. 8A-8C show a non-limiting example of a spherical visualization of cells from lupus nephritis (LN) single-cell RNA-Seq data, in accordance with disclosed embodiments, including a left 90° view (FIG. 8A), a front view (FIG. 8B), and a right 90° view (FIG. 8C). As observed in the spherical mapping, several clusters of varying densities can be clearly seen around the surface of the sphere. A subset of 20 percent of cells are shown for clarity.

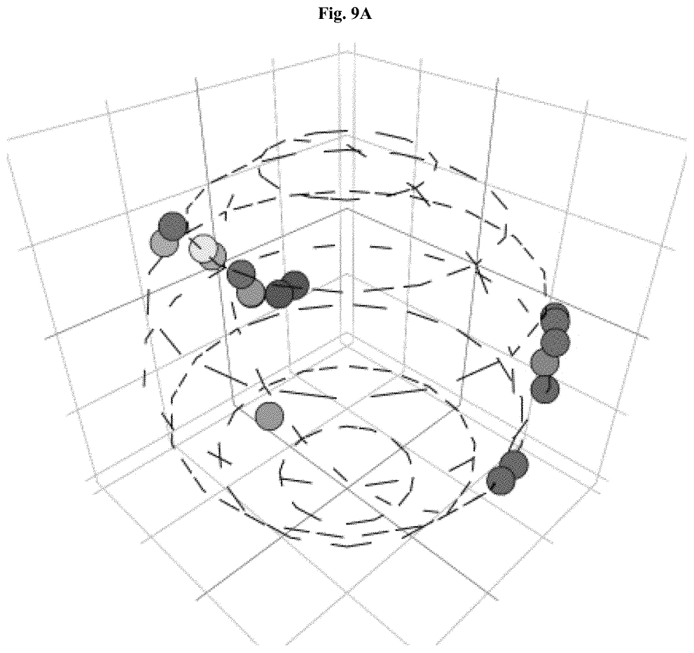

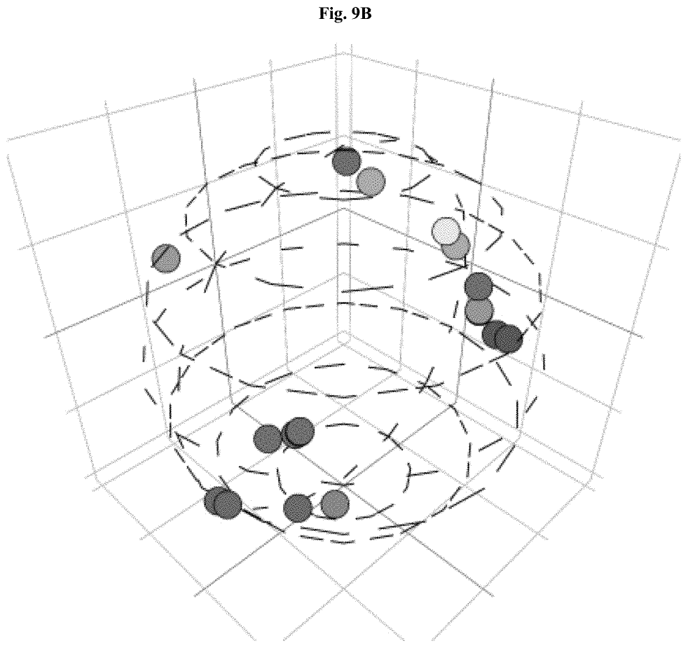

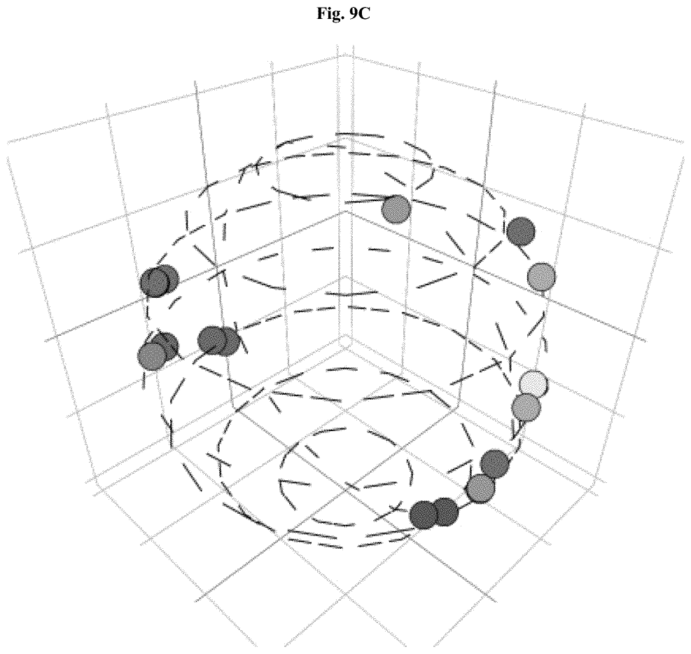

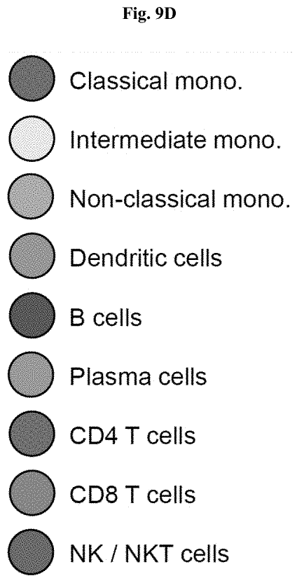

FIGS. 9A-9D show a non-limiting example of a spherical visualization of centroids of clusters identified, in accordance with disclosed embodiments, including a left 90° view (FIG. 9A), a front view (FIG. 9B), and a right 90° view (FIG. 9C) of multiple cell populations, including classical monocytes, intermediate monocytes, non-classical monocytes, dendritic cells, B cells, plasma cells, CD4 T cells, CD8 T cells, and natural killer (NK) cells/natural killer T (NKT) cells (as indicated in the legend of FIG. 9D).

As observed in the spherical mapping of centroids of clusters identified by the StarShip clustering algorithm, the clusters form three distinct superclusters: (1) B cells and myeloid cells, (2) T cells and NK cells, and (3) plasma cells. Further, monocyte subpopulations werer clustered together by function (e.g., antigen presentation) rather than cell type (e.g., classical, intermediate, non-classical, etc.). Moreover, CD8T cells and NKT cells clustered separately from NK cells.

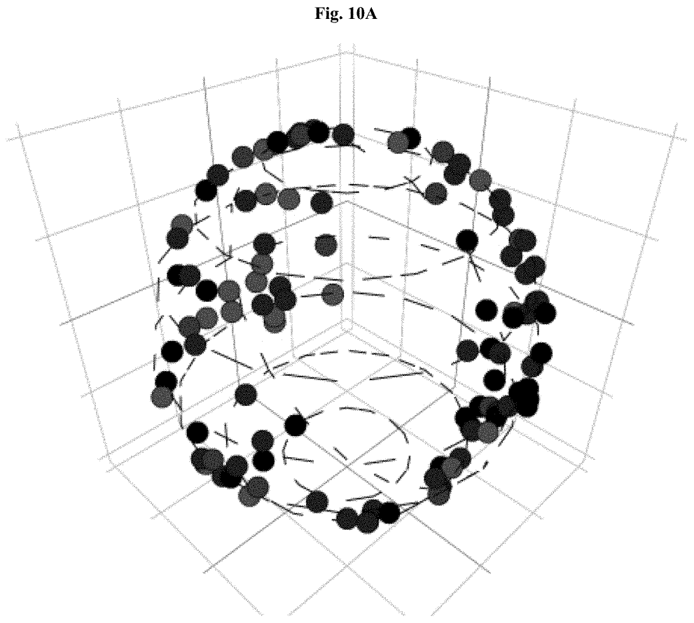

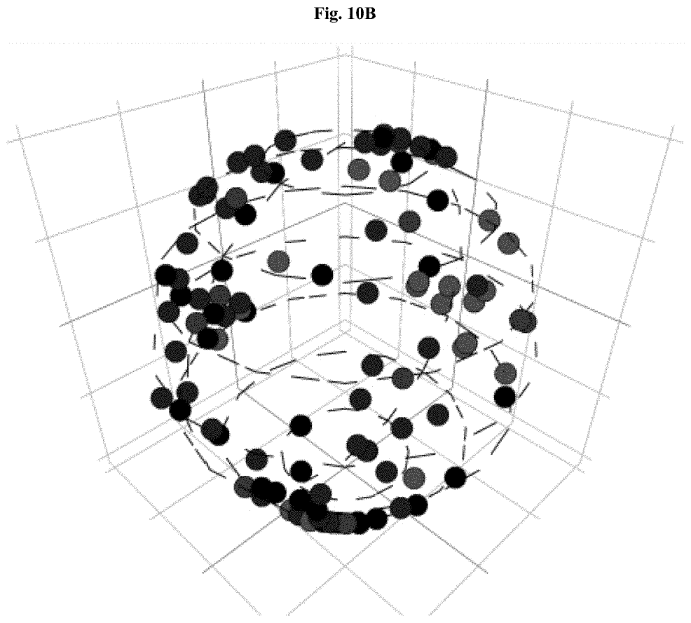

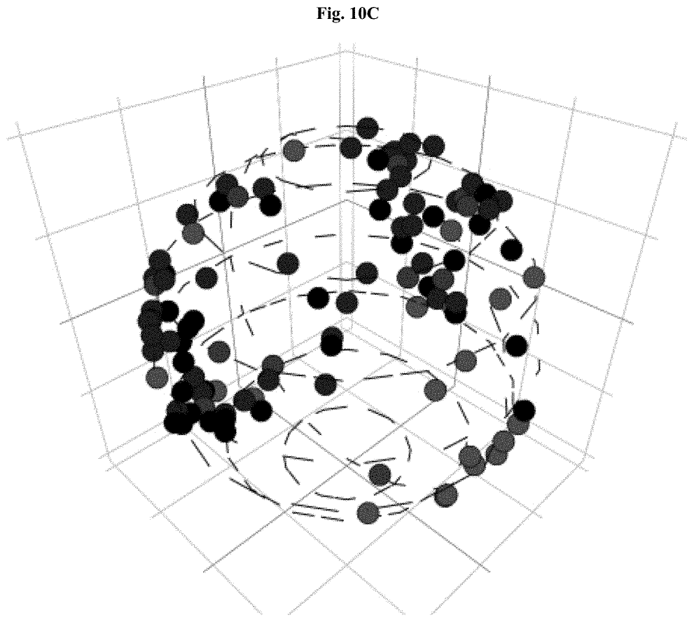

FIGS. 10A-10C show a non-limiting example of T follicular helper (Tfh) and regulatory T (Treg) signatures appearing in lupus nephritis (LN) single-cell RNA-Seq data, in accordance with disclosed embodiments, including a left 90° view (FIG. 10A), a front view (FIG. 10B), and a right 90° view (FIG. 10C). Spherical mapping was performed on a subset of CD4 T cells in order to test for Tfh and/or Treg signatures by Kolmogorov-Smirnov test. The Tfh signature comprised: BCL6, CD40LG, CD84, CXCL13, CXCR5, ICOS, MAF, PDCD1, and SH2D1A. The Treg signature comprised: FOXP3, IKZF2, RUNX3, and TIGIT. Tfh-positive cells are shown in blue, Treg-positive cells are shown in red, Tfh-positive and Treg-positive cells are shown in purple, and Tfh-negative and Treg-negative cells are shown in black.

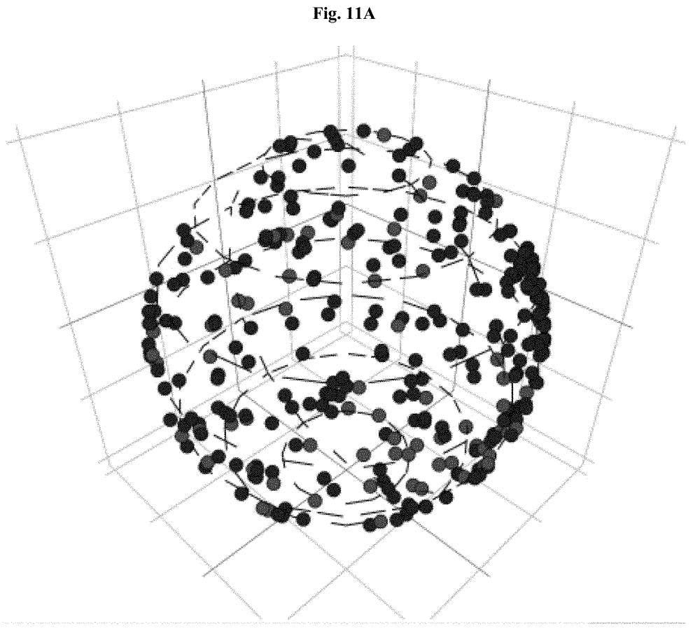

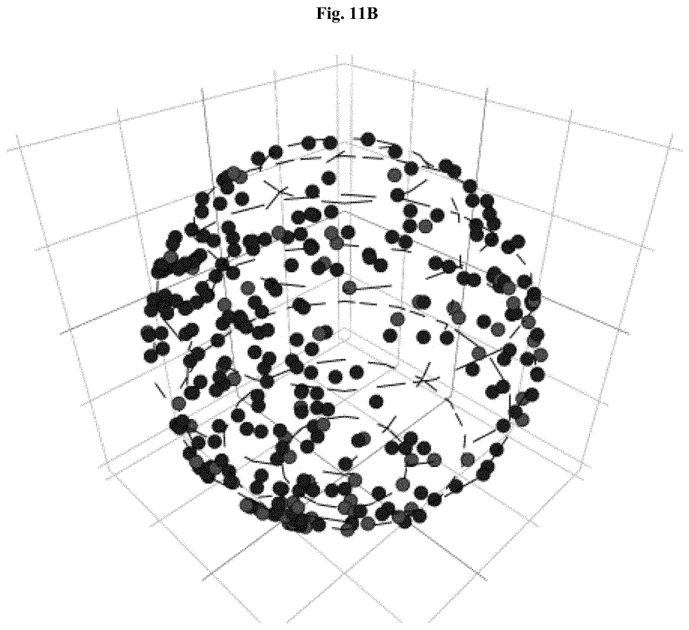

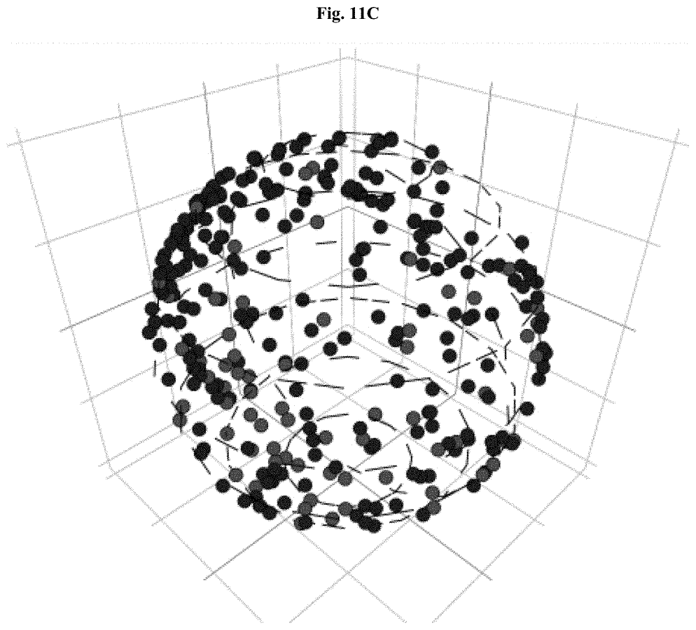

FIGS. 11A-11C shows a non-limiting example of a CD11c/T-bet systemic lupus erythematosus (SLE)_B cell signature appearing in lupus nephritis (LN) single-cell RNA-Seq data, in accordance with disclosed embodiments, including a left 90° view (FIG. 11A), a front view (FIG. 11B), and a right 90° view (FIG. 11C). Spherical mapping was performed on a subset of B cells in order to test for a CD11c hi/T-bet+ B cell signature by Kolmogorov-Smirnov test. 23 percent of B cells (71/308) were positive for the signature. The signature comprised: CD19, FCGR2A, FCRL3, FCRL5, ITGAX, MS4A1, TBX21, and TNFRSF13B (as described by, for example, Wang et al., “Supramolecular Kandinsky circles with high antibacterial activity,” Nature Communications, May 8, 2018, which is incorporated herein by reference in its entirety). Positive cells are shown in red, and negative cells are shown in blue.

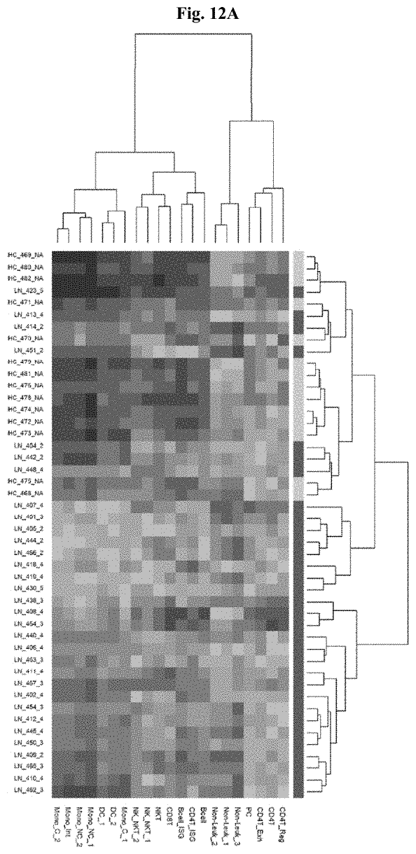

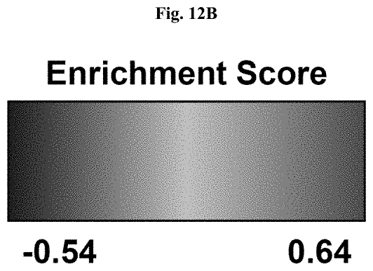

FIGS. 12A-12B show a non-limiting example of enrichment of cluster markers in lupus nephritis (LN) glomerulus (FIG. 12A) and an associated legend of enrichment score (FIG. 12B), in accordance with disclosed embodiments, including GSVA enrichment scores of cluster marker gene lists in microarray data from kidney biopsies of lupus nephritis patients (shown in purple) and healthy controls (shown in gold).

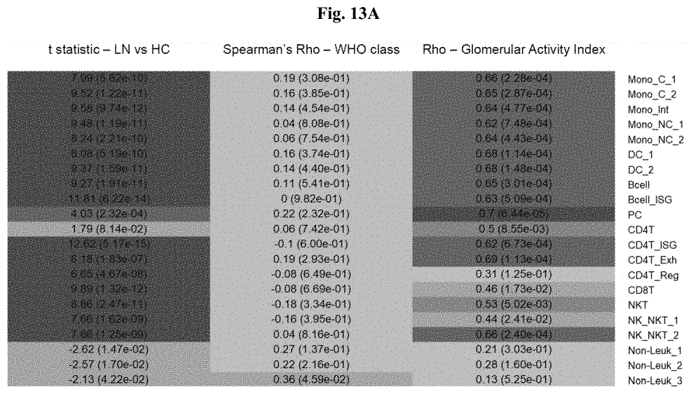

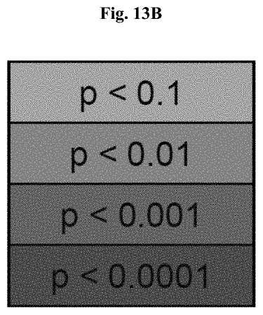

FIGS. 13A-13B show a non-limiting example of associations between enrichment and subject traits (FIG. 13A) and an associated legend of statistical significance as indicated by different ranges of p-values (FIG. 13B), in accordance with disclosed embodiments, including GSVA enrichment scores of cluster marker gene lists as compared to subject traits. The association with subject cohort (lupus nephritis (LN) vs. healthy controls (HC)) was assessed by t test. Associations with WHO kidney disease classification and glomerular activity index were assessed by Spearman rank correlation. Values are given as t statistic/Spearman's rho (p value).

Through dynamic spherical k-means, the StarShip clustering algorithm effectively groups single-cell RNA-Seq data into meaningful clusters. For example, the clustering algorithm delineated leukocyte populations from lupus nephritis biopsies into readily identifiable phenotypic categories. CD11c hi/T-bet+ B cells were detected in lupus nephritis, but they did not separate cleanly from ordinary B cells. Cluster definitions from StarShip analysis of single-cell RNA-Seq data were informative in classifying bulk lupus nephritis microarray data.

The StarShip clustering method can effectively partition unknown cells from scRNA-Seq data sets into biologically relevant clusters without using or requiring prior knowledge of the number of cell types present. The similarity between the performance of the StarShip algorithm and one-off k-means, which does incorporate this prior knowledge, highlights the value of this dynamic technique.

### Example 7—Visualization of Clusters Generated from Single-Cell RNA-Seq Data of Lupus Nephritis and Pancreas Samples

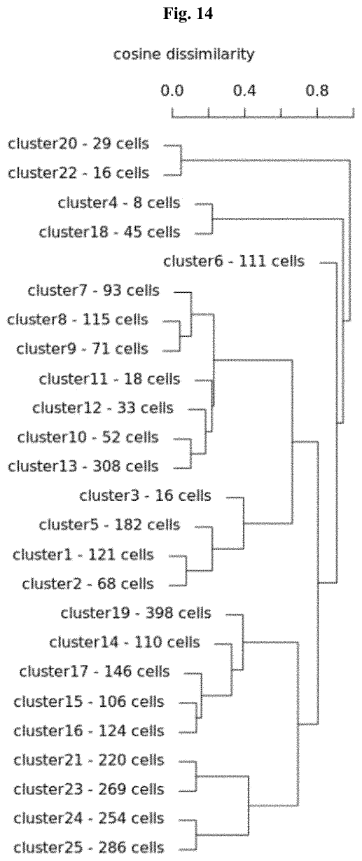

FIG. 14 shows a non-limiting example of a cluster dendrogram visualization of clusters identified by the clustering algorithm based on cosine dissimilarities between cluster centers from lupus nephritis (LN) single-cell RNA-Seq data, in accordance with disclosed embodiments. The clustering algorithm identified 25 clusters in the LN scRNA-Seq data, ranging in size from 8 cells (cluster 4) to 398 cells (cluster 19).

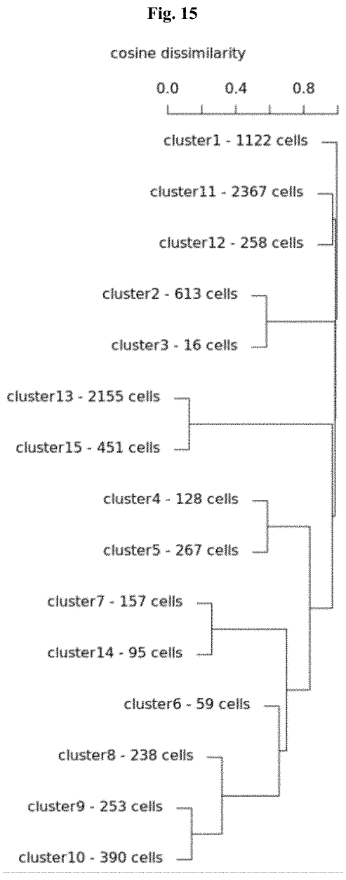

FIG. 15 shows a non-limiting example of a cluster dendrogram visualization of clusters identified by the clustering algorithm based on cosine dissimilarities between cluster centers from pancreas single-cell RNA-Seq data, in accordance with disclosed embodiments. The clustering algorithm identified 15 clusters in the panceas scRNA-Seq data, ranging in size from 16 cells (cluster 3) to 2367 cells (cluster 11).

Table 2 shows a comparison of clusters from pancreas single-cell RNA-Seq data with author-supplied cell types. Some cell types are predominantly clustered into a single cluster (e.g., acinar cells in cluster 1, activated stellate cells in cluster 5, alpha cells in cluster 11, delta cells in cluster 2, endothelial cells in cluster 7, gamma cells in cluster 12, macrophage cells in cluster 6, and Schwann cells in cluster 10). Other cell types are clustered across two clusters (e.g., beta cells in clusters 13 and 15). Still other cell types are clustered across three or more clusters (e.g., ductal cells in clusters 1, 8, 9, and 10; epsilon cells in clusters 2, 3, 8, and 11; mast cells in clusters 6, 13, and 14; and quiescent stellate cells in clusters 4, 5, and 14).

TABLE 2
Comparison of clusters from pancreas scRNA-Seq data

| | | Author-supplied cell type | | | | | | | | | | | | | |
|---|---|---|---|---|---|---|---|---|---|---|---|---|---|---|---|
| | | | Activated | | | | | Endo- | | | Macro- | | Quiescent | | T |
| | | Acinar | stellate | Alpha | Beta | Delta | Ductal | thelial | Epsilon | Gamma | phage | Mast | stellate | Schwann | cell |
| Cluster | 1 | 949 | 3 | 6 | 1 | 2 | 158 | 1 | 0 | 1 | 0 | 0 | 1 | 0 | 0 |
| assignment | 2 | 0 | 2 | 2 | 9 | 583 | 11 | 0 | 3 | 0 | 0 | 0 | 3 | 0 | 0 |
| | 3 | 0 | 2 | 0 | 0 | 2 | 1 | 2 | 4 | 0 | 0 | 1 | 4 | 0 | 0 |
| | 4 | 1 | 40 | 0 | 0 | 0 | 0 | 14 | 0 | 0 | 1 | 0 | 71 | 1 | 0 |
| | 5 | 0 | 215 | 0 | 1 | 0 | 0 | 10 | 0 | 0 | 1 | 0 | 40 | 0 | 0 |
| | 6 | 0 | 0 | 0 | 1 | 0 | 0 | 0 | 0 | 0 | 41 | 14 | 1 | 0 | 2 |
| | 7 | 0 | 0 | 0 | 1 | 0 | 0 | 155 | 0 | 0 | 0 | 0 | 1 | 0 | 0 |
| | 8 | 0 | 0 | 0 | 0 | 1 | 229 | 0 | 6 | 1 | 0 | 0 | 1 | 0 | 0 |
| | 9 | 0 | 1 | 1 | 1 | 1 | 247 | 0 | 0 | 0 | 1 | 0 | 0 | 1 | 0 |
| | 10 | 4 | 1 | 1 | 0 | 1 | 370 | 0 | 0 | 0 | 2 | 0 | 0 | 8 | 3 |
| | 11 | 1 | 6 | 2302 | 12 | 2 | 24 | 4 | 4 | 3 | 4 | 0 | 5 | 0 | 0 |
| | 12 | 0 | 0 | 4 | 2 | 0 | 2 | 1 | 0 | 247 | 0 | 1 | 1 | 0 | 0 |
| | 13 | 3 | 12 | 10 | 2056 | 9 | 30 | 16 | 1 | 0 | 4 | 4 | 8 | 1 | 1 |
| | 14 | 0 | 1 | 0 | 2 | 0 | 1 | 47 | 0 | 1 | 0 | 4 | 36 | 2 | 1 |
| | 15 | 0 | 1 | 0 | 439 | 0 | 4 | 2 | 0 | 2 | 1 | 1 | 1 | 0 | 0 |

While preferred embodiments have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the scope of the disclosure. It should be understood that various alternatives to the embodiments described herein may be employed in practice. Numerous different combinations of embodiments described herein are possible, and such combinations are considered part of the present disclosure. In addition, all features discussed in connection with any one embodiment herein can be readily adapted for use in other embodiments herein. It is therefore contemplated that the invention shall also cover any such alternatives, modifications, variations or equivalents. It is intended that the following claims define the scope of the disclosure and that methods and structures within the scope of these claims and their equivalents be covered thereby.

1. A computer-implemented method for clustering cells using gene differential expression of single cells, the method comprising:
a) mapping RNA-Seq data of a plurality of cells onto a sphere, wherein the sphere has a dimensionality based on the RNA-Seq data of the plurality of cells;
b) calculating a plurality of distances, wherein each of the plurality of distances is associated with an angle between two different cells mapped onto the sphere;
c) clustering the plurality of cells into two clusters based on the plurality of distances;
d) evaluating each of the two clusters using a pre-determined stopping criterion; and
e) repeating c) and d) on each of the two clusters until the pre-determined stopping criterion or a second stopping criterion is met.

2. The method of claim 1, wherein the RNA-Seq data comprises data entries of gene expression levels.

3. (canceled)

4. (canceled)

5. The method of claim 1, wherein the RNA-Seq data comprises data of each single cell of the plurality of cells.

6. The method of claim 1, wherein the RNA-Seq data of one or more cells of the plurality of cells comprise data entries that are identical to the data entries in other cells of the plurality of cells.

7. (canceled)

8. The method of claim 1, wherein the sphere is a unit hypersphere.

9. The method of claim 1, wherein the dimensionality of the sphere is based on a number of genes in the RNA-Seq data.

10. (canceled)

11. The method of claim 1, wherein mapping the RNA-Seq data of the plurality of cells onto the sphere is based on gene expression levels.

12. The method of claim 1, wherein mapping the RNA-Seq data of the plurality of cells onto the sphere comprises normalization of the RNA-Seq data of each of the plurality of cells by a corresponding Euclidean length thereof.

13. (canceled)

14. (canceled)

15. (canceled)

16. (canceled)

17. The method of claim 1, wherein the pre-determined stopping criterion comprises a minimum cluster size, a number of genes that are differently expressed between two different clusters, a clustering silhouette, or a combination thereof.

18. (canceled)

19. (canceled)

20. (canceled)

21. The method of claim 1, further comprising, prior to a), filtering one or more pre-determined genes from the RNA-Seq data, filtering one or more genes from the RNA-Seq data based on expression levels thereof, filtering one or more genes from the RNA-Seq data based on detection rates thereof in the plurality of cells, or filtering one or more cells from the RNA-Seq data based on a number of RNA-Seq transcripts detected, a number of genes detected, or proportion of mitochondrial transcripts detected.

22. (canceled)

23. (canceled)

24. (canceled)

25. The method of claim 1, further comprising visualizing the two clusters in c), e), or both on a three-dimensional sphere.

26. The method of claim 1, further comprising determining one or more genes that distinguish different clusters or different groups of clusters, subsequent to e).

27. (canceled)

28. (canceled)

29. (canceled)

30. The method of claim 1, wherein the RNA-Seq data of the plurality of cells is not normalized, prior to a); wherein the RNA-Seq data of the plurality of cells is not subjected to imputation, prior to a); or wherein the method does not use a priori knowledge of a number of clusters of the plurality of cells.

31. (canceled)

32. (canceled)

33. (canceled)

34. (canceled)

35. The method of claim 1, further comprising, after subsequent to e), identifying a cell type from among the two clusters, wherein the cell type is classical monocytes, intermediate monocytes, non-classical monocytes, dendritic cells, B cells, T cells, plasma cells, CD4 T cells, CD8 T cells, NK cells, or NKT cells.

36. (canceled)

37. The method of claim 35, further comprising identifying a number of cells of the cell type.

38. (canceled)

39. The method of claim 35, further comprising determining a p-value for the identification of the cell type.

40. The method of claim 1, wherein the plurality of cells is obtained from a biological sample of a subject, wherein the biological sample is obtained from an organ of the subject, and wherein the organ is a kidney, pancreas, liver, lung, heart, brain, large intestine, small intestine, gallbladder, bile duct, spleen, bladder, prostate, testis, ovary, cervix, lymph node, adrenal gland, salivary gland, bone marrow, or skin.

41. (canceled)

42. (canceled)

43. (canceled)

44. (canceled)

45. The method of claim 40, further comprising identifying a presence or absence of a disease or disorder of the subject based on the identified clusters, wherein the disease or disorder is systemic lupus erythematosus (SLE), lupus nephritis (LN), LN glomerulus, or LN tubulointerstitium.

46. (canceled)

47. The method of claim 45, further comprising determining a kidney disease classification or a glomerular activity index of the subject based on the identified clusters.

48. (canceled)

49. The method of claim 45, wherein the identified clusters comprise one or more of: leukocytes, T follicular helper (Tfh)-positive cells, T follicular helper (Tfh)-negative cells, regulatory T (Treg)-positive cells, regulatory T (Treg)-negative cells, T-bet-positive cells, and T-bet-negative cells.

50.-150. (canceled)
